(19) United States
(12) Patent Application Publication
(10) Pub. No.: US 2013/0115637 A1
(43) Pub. Date: May 9, 2013

(54) Efficiency of Prion Conversion in vitro and Sensitivity of Prion Detection

(71) Applicants: Ilia V. Baskakov, Columbia, MD (US); Robert G. Rohwer, Ellicott City, MD (US); Nuria Gonzalez-Montalban, Baltimore, MD (US); Natallia Makarava, Baltimore, MD (US)

(72) Inventors: Ilia V. Baskakov, Columbia, MD (US); Robert G. Rohwer, Ellicott City, MD (US); Nuria Gonzalez-Montalban, Baltimore, MD (US); Natallia Makarava, Baltimore, MD (US)

(73) Assignees: The United States of America as represented by the Dept. of Veterans Office of General Counsel, Washington, DC (US); UNIVERSITY OF MARYLAND, BALTIMORE, Baltimore, MD (US)

(21) Appl. No.: 13/808,860

(22) PCT Filed: Jul. 8, 2011
(86) PCT No.: PCT/US2011/043387

§ 371 Date: Jan. 7, 2013

Related U.S. Application Data
(60) Provisional application No. 61/362,477, filed on Jul. 8, 2010.
(60) Provisional application No. 61/486,559, filed on May 16, 2011.

(51) Int. Cl. G01N 33/68

(52) U.S. Cl. CPC G01N 33/6896
USPC 435/7.92; 436/501; 435/287.2; 422/430

(57) ABSTRACT

The present invention relates to improved methods and kits for the amplification detection of pathogenic prion proteins in samples. In some aspects of the invention, the method comprises: i) contacting the sample with a source of PrP^{C} to make a reaction mixture; ii) incubating the reaction mixture; iii) agitating the reaction mixture of (ii) in the presence of one or more beads; and iv) detecting the amplified PrP^{Sc}. The present invention also provides methods of screening to identify agents that modulate PrP^{Sc} formation, by carrying out the methods of the invention in the presence and absence of the agent, and comparing the amount of PrP^{Sc} generated between the samples. The present methods can be employed to obtain quantities of PrP^{Sc} for structural or other studies.

### CROSS-REFERENCE TO RELATED APPLICATIONS

This application claims the benefit of U.S. Provisional Appl. No. 61/486,559 filed May 16, 2011, and U.S. Provisional Appl. No. 61/362,477 filed Jul. 8, 2010. The content of the aforesaid applications are relied upon and are incorporated by reference herein in their entireties.

### STATEMENT OF FEDERALLY SPONSORED RESEARCH AND DEVELOPMENT

This invention was made with government support under NIH Grant No, NS045585 awarded by the National Institutes of Health. The government has certain rights in the invention.

### FIELD OF THE INVENTION

The field of the invention relates generally to prion disease and methods for amplifying and detecting prions, in particular, prion detection by protein misfolding cyclic amplification assays.

### BACKGROUND OF THE INVENTION

Protein misfolding cyclic amplification (PMCA) provides faithful amplification of mammalian prions in vitro and, since its introduction in 2001, has become an important tool in prion research (Saborio et al. Nature 411: 810-813). To date, PMCA provides the most sensitive approach for detecting miniscule amounts of prion infectivity, including detection of prions in blood or peripheral tissues at preclinical stages of the disease (Saa et al. J Biol Chem 281:35245-35252 (2006); Gonzalez-Romero et al. FEBS Lett 582: 3161-3166 (2008); Murayama et al. J Gen Virol 88: 2890-2898 (2007); Shikiya et al. J Virol 84: 5706-5714 (2010); Saa et al. Science 313: 92-94 (2006); Tattum M H, Jones S, Pal S, Collinge J, Jackson G S (2010) Discrimination between prion-infected and normal blood samples by protein misfolding cyclic amplification. Transfusion (in press); Haley et al. Plos ONE 4:e7990 (2009)). In recent studies, PMCA was employed for generating infectious prions (PrP^{Sc}) in vitro de novo in crude brain homogenate, and for producing infectious prions from the cellular prion protein (PrP^{C}) purified from normal mammalian brains and recombinant PrP (rPrP) produced in E. coli (Barris et al, PLOS Pathog 5: e1000421 (2009); Deleault et al. Proc Acad Natl Sci USA 104: 9741-9746 (2007); Wang et al. Science 327: 1132-1135 (2010)). Furthermore, PMCA has been used for identifying cofactors that are involved in prion replication and assessing the impact of glycosylation on replication of prion strains (Deleault et al. J Biol Chem 280: 26873-26879 (2005); Deleault et al, Biochemistry 49: 3928-3934 (2010); Deleault et al. Nature 425: 717-720 (2003); Nishina et al. Biochemistry 45: 14129-14139 (2006); Mays C E, Ryou C, Plasminogen stimulates propagation of protease-resistant prion protein in vitro, Faseb J 24: 5102-5112 (2010)). PMCA has also been utilized for assessing the prion transmission barrier, prion interference and adaptation to new hosts (Castilla et al. Cell 134: 757-768 (2008); Green et al. PLOS Pathog 4: e1000139 (2008); Shikiya et al. J Virol 84: 5706-5714 (2010); Meyerett et al. Virology 382: 267-276 (2008)).

PMCA reactions comprise two alternating steps: incubation and sonication. Sonication fragments PrP^{Sc} particles or fibrils into smaller pieces, a process that that is believed to result in the multiplication of active centers of PrP^{Sc} growth. During the incubation step, small PrP^{Sc} particles grow by recruiting and converting PrP^{C} molecules into PrP^{Sc}.

While the discovery of PMCA has provided new opportunities for exploring the prion replication mechanism, the low yield of PrP^{Sc} has limited its utility for structural studies. It also implies that only a small sub-fraction of PrP^{C} may be available for conversion. Furthermore, the efficiency of amplification in PMCA varies dramatically depending on minor variations in experimental parameters, including those that are difficult to control, such as the age of the sonicator's horn and individual patterns of horn corrosion. Previous strategies for improving the efficiency of PMCA focused on increasing the number of cycles within a single PMCA round or increasing the substrate concentration by using a normal brain homogenate (NBH) from transgenic mice overexpressing PrP^{C} (Saa et al. J Biol Chem 281: 35245-35252 (2006); Mays et al. Biochem Biophys Res Commun 388: 306-310 (2009); Kurt et al. J Virol 81: 9605-9608 (2007)).

This background information is provided for the purpose of making available, information believed by the applicant to be of possible relevance to the present invention. No admission is necessarily intended, nor should it be construed, that any of the preceding information constitutes prior art against the present invention.

There exists a need for new and improved methods for detecting prions in samples that are more sensitive and reliable, and can be practically achieved to enable early detection of prion related diseases. There further exists a need for rapid and reliable methods of amplifying prions to serve as substrates for the study and analysis of prions.

### SUMMARY OF THE INVENTION

In one aspect, the invention provides a method for amplifying and detecting PrP^{Sc} in a sample, comprising: i) contacting the sample with a source of PrP^{C} to make a reaction mixture; ii) incubating the reaction mixture; iii) agitating the reaction mixture of (ii) in the presence of one or more beads; and iv) detecting the amplified PrP^{Sc}. In some embodiments, steps (ii) and (iii) are repeated between 1 and 500 times before step iv) is conducted. In some embodiments, a portion of the reaction mixture is diluted and added to additional PrP^{C} after a number of steps of ii) and iii) have been performed, and steps ii) and iii) are repeated one or more times with the diluted sample. In some embodiments, the reaction mixture is agitated by sonication.

In some embodiments, the reaction mixture is incubated for a period of time selected from the group consisting of: approximately 5 minutes, approximately 10 minutes, approximately 15 minutes, approximately 20 minutes, approximately 25 minutes, approximately 30 minutes, approximately 35 minutes, approximately 40 minutes, approximately 45 minutes, approximately 50 minutes, approximately 55 minutes, approximately 60 minutes, approximately 75 minutes, approximately 90 minutes and approximately 120 minutes.

In some embodiments, the one or more beads are made from a substance selected from the group consisting of: one or more polymeric substances, polytetrafluoroethylene (PTFE; TEFLON), stainless steel, neoprene, nylon, ethylene propylene diene monomer (EPDM), nitrile rubber, zytel nylon, acetal, glass, ceramic, polypropylene and polystyrene.

In some embodiments, the beads are at least a size selected from the group consisting of about 0.1 mm, about 0.2 mm, about 0.3 mm, about 0.4 mm, about 0.5 mm, about 0.6 mm, about 0.7 mm, about 0.8 mm, about 0.9 mm, about 1 mm, about 1.1 mm, about 1.2 mm, about 1.3 mm, about 1.4 mm, about 1.5 mm, about 1.59 mm, about 1.6 mm, about 1.7 mm, about 1.8 mm, about 1.9 mm, about 2.0 mm, about 2.1 mm, about 2.2 mm, about 2.3 mm, about 2.38 mm, about 2.4 mm, about 2.5 mm, about 2.6 mm, about 2.7 mm, about 2.8 mm, about 2.9 mm, about 3.0 mm, about 3.5 mm, about 4.0 mm, about 4.5 mm and about 5.0 mm.

In some embodiments, the number of beads is selected from the group consisting of one bead, 2 beads, 3 beads, 4 beads, 5 beads, 6 beads, 7 beads, 8 beads, 9 beads, 10 beads, 11 beads, 12 beads, 13 beads, 14 beads, 15 beads, 16 beads, 17 beads, 18 beads, 19 beads, 20 beads, 21 beads, 22 beads, 23 beads, 24 beads and 25 beads. In some embodiments, the number of beads is greater than 25. For example, 30 beads, 40 beads, 50 beads, 60 beads, 70 beads, 80 beads, 90 beads, 100 beads, 150 beads, 200 beads, 250 beads or more can be used. In some embodiments, more than 25 beads are employed when the size of the bead is small, for example, 0.9 mm or less.

In some embodiments, the volume occupied by the one or more beads in the reaction mixture is about 5% of the total volume. In some embodiments, the beads occupy about 10%, about 15%, about 20%, about 25%, about 30%, about 35%, about 40%, about 45%, about 50%, about 55%, about 60%, about 65%, about 70%, or about 75% or more of the reaction mixture volume.

In some embodiments, the fold amplification of PrP^{Sc} achieved in the presence of the one or more beads is about 50-fold to about 1000-fold greater than amplification without the bead(s) present.

In some embodiments, the source of PrP^{C} is from an origin selected from the group consisting of: human, bovine, ovine, hamster, rat, mouse, canine, feline, goat, cervid, and non-human primate.

In some embodiments, the PrP^{C} is from a source selected from the group consisting of a tissue sample (whole tissue, homogenate or fraction thereof), a bodily fluid sample, cell lysate from cultured cells, a recombinant source and a transgenic animal. In some embodiments, the source of PrP^{C} is normal brain homogenate.

In some embodiments, the sample is from an organism suspected of, at risk of, or known to have a prion disease, wherein the prion disease is selected from the group consisting of: scrapie (typical and atypical forms) in sheep, bovine spongiform encephalopathy (BSE; also known as mad cow disease, including classical and the atypical forms BSE-H and BSE-L) in cows, bovine amyloidotic spongiform encephalopathy (BASE) in cows, transmissible mink encephalopathy (TME) in mink, chronic wasting disease (CWD) in elk, moose, or deer, feline spongiform encephalopathy, ungulate encephalopathy in nyala, oryx or greater kudu, and Creutzfeldt-Jakob disease (CJD) and its varieties (including but not limited to iatrogenic Creutzfeldt-Jakob disease (iCJD), variant Creutzfeldt-Jakob disease (vCJD), genetic Creutzfeldt-Jakob disease (ftM), and sporadic Creutzfeldt-Jakob disease (sCJD)), Gerstmann-Straussler-Scheinker syndrome (GSS), fatal familial insomnia (fFI), sporadic fatal insomnia (sFI), kuru, and Alpers syndrome in humans. In some embodiments, the detection of PrP^{Sc} indicates the presence of a prion disease.

In some embodiments, the sample is from an organism selected from the group consisting of human, bovine, cervids, sheep, primate and rodent.

In some embodiments, the sample is a tissue sample (whole tissue, homogenate or fraction thereof) or other sample of bodily origin including, but not limited to, blood, lymph nodes, brain tissue (includes whole brain, anatomical parts, or fractions and homogenates thereof), spinal cord, tonsils, internal organs (such as spleen, stomach, pancreas, liver, intestine (large or small), lungs, heart, thymus, bladder or kidney), skin, muscle, appendix, olfactory epithelium, nasal tissue, cerebral spinal fluid, urine, feces, milk, mucosal secretions, tears and/or saliva.

In another aspect, the invention provides methods of screening to identify an agent that modulates PrP^{Sc} formation, comprising: i) contacting a sample having PrP^{Sc} with a source of PrP^{C} to make a reaction mixture; ii) incubating the reaction mixture in the presence and absence of the agent; iii) agitating the reaction mixture of (ii) in the presence of one or more beads; and iv) detecting and comparing the level of the amplified PrP^{Sc} generated in the presence and absence of the agent.

In another aspect, the invention provides kits for the amplification and detection of PrP^{Sc}. In some embodiments, the kit comprises in suitable container, one or more beads, a source of PrP^{C}, and optionally further comprises one or more of the following components: 1) a reaction mixture buffer; 2) decontamination solution; 3) a positive control sample containing PrP^{Sc}; 4) a negative control sample that does not contain PrP^{Sc}; 5) one or more proteases, such as proteinase K; and 6) one or more reagents for the detection of PrP^{Sc}.

### BRIEF DESCRIPTION OF THE FIGURES

FIG. 1. Beads improve the yield and rate of PrP^{Sc} conversion. (A) 263K scrapie brain material was diluted 10³-fold into 10% normal brain homogenate (NBH) and subjected to 48 PMCA cycles in the absence of beads (lane 1) or presence of 1, 2, 3, 4 or 5 small beads (S, lanes 2-6) or 1, 2 or 3 large beads (L, lanes 7-9). 10% NBH loaded at 100%, 75%, 50% or 25% amounts were used to estimate the amplification yield (lanes 10-13, respectively). (B) 263K scrapie brain material was diluted 10³-fold into 10% NBH and subjected to 4, 8, 16, 24 or 48 PMCA cycles in the absence of beads (lanes 2-6) or presence of 5 small beads (lanes 7-11). Prior to electrophoresis, samples were digested with Proteinase K (PK) as indicated.

FIG. 2. Bioassay of PMCA products. Incubation time to terminal disease stage in individual animals (circles) and in animal groups represented as mean±S.D. (black bars). Animals were inoculated with 10⁻⁴-fold diluted 263K brain homogenate (group 1); 10⁻⁴-fold diluted 263K brain homogenate sonicated for 48 cycles (a single PMCA round) in the absence of beads (group 2) or presence of 3 large beads (group 3) in the absence of NBH; and 10⁻⁴-diluted 263K brain homogenate subjected to six serial PMCA rounds in the absence of beads (group 4) or presence of 3 large beads (group 5) using 1:10 dilutions between rounds. After serial PMCA, the amplification products were diluted an additional 10-fold to obtain 10¹⁰-fold dilutions of the original seeds for inoculating groups 4 and 5.

FIG. 3. Beads improve the sensitivity of PrP^{Sc} detection. (A) 263K scrapie brain material was serially diluted 10⁴-, 10⁵-, 10⁶- or 10⁷-fold into 10% NBH, subjected to 48 PMCA cycles in the absence of beads (lanes 3,5) or presence of five small beads (lanes 4,6,7,8) and digested with PK. Undigested 10% NBH (lanes 1,2) was loaded as a reference. (B) PrP^{Sc} amplification in 48 cycles of PMCA conducted with five small beads for 263K scrapie brain material serially diluted 10⁴-, 10⁵- or 10⁶-folds as analyzed by dot blotting. The data represent average and standard deviation of three independent PMCA reactions. The amplification fold was calculated using calibration curve in FIG. 12.

FIG. 4. Amplification of minute amounts of PrP^{Sc} in PMCAb. (A) 263K brain material was serially diluted 10¹⁰-, 10¹²-, or 10¹⁴-fold and subjected to sPMCAb amplification in the presence of 3 large beads for 6 rounds as indicated (each round consists of 48 sonication cycles, 10-fold dilutions were used for subsequent rounds). Amplification in three independent experiments (A, B, and C) are shown. (B) Up to six rounds of PMCAb in non-seeded NBHs (reactions A and B) or in NBHs seeded with 10 μl of 10% NBH prepared from 661 days old Syrian Hamsters (reactions C and D) were performed as negative controls. Undigested 10% NBH is provided as a reference.

FIG. 5. Beads improve the amplification efficiency of SSLOW. SSLOW scrapie brain material was diluted 10³-fold (lanes 1-8) or 10⁴-fold (lanes 9-15) into 10% NBH, subjected to serial PMCA in the absence or presence of 3 small beads, as indicated, and digested with PK. Each PMCA round consisted of 48 cycles; the material amplified in each round was diluted 10-fold into 10% NBH for the next PMCA round. Undigested 10% NBH (lane 1) loaded at 1/10^{th} the amount of the digested samples is provided as a reference.

FIG. 6. Beads counteract the negative effect of rPrP on PrP^{Sc} amplification. 263K scrapie brain material was diluted 10³-fold into 10% NBH and subjected to a serial PMCA in the absence or presence of α-rPrP (5 μg/ml) and absence or presence of 3 small beads, as indicated. Each PMCA round consisted of 48 cycles; the material amplified in each round was diluted 10-fold into 10% NBH for the next PMCA round. Undigested 10% NBH (lane 1) loaded at 1/10^{th} the amount of the digested samples is provided as a reference.

FIG. 7. PMCA with beads preserves species barrier. (A) SSLOW scrapie brain material was diluted 10³-fold into 10% hamster or 10% mouse NBH, subjected to serial PMCA in the absence or presence of 3 large beads, as indicated, and digested with PK. Undigested 10% NBH (lane 1) is provided as a reference, (B) 263K scrapie brain material was diluted 10³-fold into 10% hamster or 10% mouse NBH, subjected to serial PMCA in the absence or presence of 3 large beads, as indicated, and digested with PK. Undigested 10% NBH (lane 1) is provided as a reference. Each PMCA round consisted of 48 cycles; the material amplified in each round was diluted 10-fold into 10% NBH for the next PMCA round.

FIG. 8. Effect of bead material on efficiency of amplification. (A) 263K scrapie brain material was serially diluted 10⁵-fold into 10% hamster NBH and subjected to 48 PMCA cycles in the presence of two beads made from Teflon (purchase from McMaster-Carr—lane 2, or Small Parts—lane 3), stainless steel 440C, neoprene, nylon, EPDM, nitrile, stainless still 302, or acetal, as indicated. Prior to electrophoresis, samples in lanes 2-10 were digested with PK. Undigested 10% hamster NBH (lane 1) was loaded as a reference. Without PMCA, 10⁵-fold diluted 263K brain material was not detectable by Western blotting (not shown). (B) RML scrapie brain material was serially diluted 10⁴-fold into 10% mouse NBH and subjected to 48 PMCA cycles in the absence of beads (lane 3) or presence of two beads made from Teflon (purchase from Small Parts—lane 4), neoprene, nylon, EPDM, nitrile, stainless still 302, or acetal, as indicated. Prior to electrophoresis, samples in lanes 2-10 were digested with PK. Undigested 10% mouse NBH (lane 1) was loaded as a reference.

FIG. 9. AFM imaging of rPrP fibril fragmentation. AFM imaging of intact rPrP fibrils (A) and fibrils sonicated for 30 sec in the absence (B) or presence of 5 small beads (C) using sonication conditions identical to those used in PMCA. Scale bars=0.5 μm. (D) Analysis of length, width and height for intact rPrP fibrils (green circles) and fibrils sonicated in the absence (orange circles) or presence of 5 small beads (red circles).

FIG. 10. Proteinase K assay of scrapie brain homogenates. Western blotting of scrapie brain homogenates from animal groups #2, 3, 4 and 5. Two brain homogenates per group are shown. 10% brain homogenates were treated with 20 μg/ml PK for 30 min at 37° C., 3F4 antibody was used for western blotting.

FIG. 11. PMCAb does not produce PrP^{Sc} de novo. 10% NBH was subjected to a three rounds of serial PMCAb (with 3 large heads) in the absence of seeds and digested with PK. Each PMCA round consisted of 48 cycles; 10-fold dilutions were used for serial rounds. 32 independent reactions were analyzed. Undigested 10% NBH (lane 1) are showed as references.

FIG. 12. Quantitative estimates of PrP^{Sc} amplification fold. (A) 263K scrapie brain material was serially diluted into 10% NBH to the final concentrations of scrapie brain ranging from 10% to 0.0625%, then digested with 50 μg/ml PK for 1 h at 37° C. and analyzed using a 96-well dot blot. The signal intensity was measured using a Typhoon 9200 Variable Mode Imager and was found to be linear within the concentrations of scrapie brain homogenate from 0.0625% to 1% as shown in panel B. This concentration range was used to estimate the fold amplification of PrP^{Sc} in PMCA.

FIG. 13, Analysis of PrP^{Sc} fold amplification in serial PMCA. 263K brain material was diluted 10⁴-fold into 10% NBH (lane 2) and subjected to a three rounds of PMCA in the absence of beads (top panel) or presence of 3 large beads (bottom panel) and digested with PK. The material amplified in each round was diluted 10-, 20-, 100-, or 1000-fold into 10% NBH for the next PMCA round, as indicated. Each PMCA round consisted of 48 cycles. Undigested 10% NBH (lane 1) is provided as a reference.

FIG. 14. Correlation of prion infectivity titer by end-point titration bioassay with PMCAb activity. Brain homogenate materials from Syrian Hamster infected with SSLOW (A) or 263K (B) were subjected to 10-fold serial dilution, then each dilution was analyzed by animal bioassay or serial PMCAb. Percentage of animals infected with prions (squares) or giving positive PMCAb reactions (triangles) is presented as a function of dilution. The solid curves represent the results of nonlinear least-square best fit of the data to sigmoidal function and the blue curves represent 95% confidence intervals. ID₅₀ and PMCAb₅₀ values were calculated from the results of fitting.

FIG. 15 (A) Schematic representation of the branched chain mechanism of prion amplification. Multiplication of active centers occurs as a result of fragmentation of PrP^{Sc} particles. Deactivation of PrP^{Sc} particles occurring due to oxidative modification, unproductive aggregation or other factors partially counteracts the process of their multiplication. (B) PrP^{Sc} amplification fold as a function of PMCA cycles calculated for reaction conducted in the presence of beads (r=1.144, this study) or absence of beads (r=1.081, data from Saa et al. J. Biol. Chem. 281:35245-35252 (2006)).

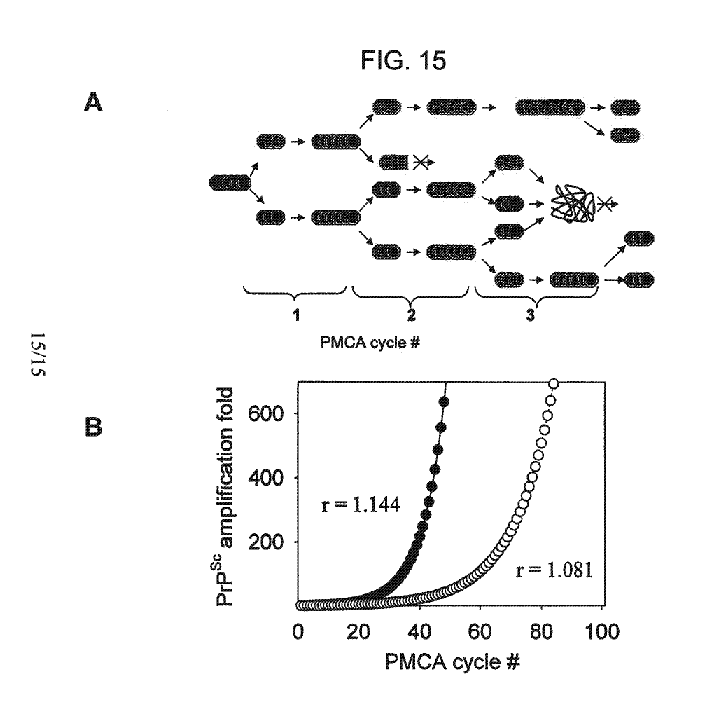

### DETAILED DESCRIPTION

The present invention is based on the surprising discovery that incorporating one or more beads in a PMCA method (referred to as PMCAb) results in remarkable improvements in the yield, rate and efficiency of prion conversion. The invention significantly improves the sensitivity of prior detection while simultaneously reducing the time required for detection of small amounts of infectious prions. This modification of the PMCA format enables fast and efficient production of high quantities of PrP^{Sc}. The results presented herein also show that the low yield observed previously has not been due to a lack of PrP^{C} susceptible to conversion, nor has it been limited by cellular cofactors. Thus, the present disclosure enables high throughput, accurate and sensitive screening of samples, as well as diagnosis of clinical disease, and further enables screening for agents that modulate PrP^{Sc} formation. In addition, the present methods can be employed to obtain quantities of PrP^{Sc} for structural or other studies.

Unless otherwise noted, technical terms are used according to conventional usage. Definitions of common terms in molecular biology may be found, for example, in Benjamin Lewin, Genes VII, published by Oxford University Press, 2000 (ISBN 019879276X); Kendrew et al. (eds.); The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341); and other similar technical references.

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). The use of “or” means “and/or” unless stated otherwise. The use of “a” herein means “one or more” unless stated otherwise or where the use of “one or more” is clearly inappropriate. The use of “comprise,” “comprises,” “comprising,” “include,” “includes,” and “including” are interchangeable and not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of.”

As used herein “about” and “approximately” mean±10% of the value indicated.

As used herein, a “priori” is a type of infectious agent composed mainly of protein. Prions cause a number of diseases in a variety of animals, including bovine spongiform encephalopathy (BSE, also known as mad cow disease) in cattle and Creutzfeldt-Jakob disease in humans. Prions are believed to infect and propagate by refolding abnormally into a structure that is able to convert normal molecules of the protein into the abnormally structured form. Most, if not all, known prions can polymerize into aggregated form rich in tightly packed beta sheets. This altered structure renders them unusually resistant to denaturation by chemical and physical agents, making disposal and containment of these particles difficult.

In prion diseases, the pathological, protease-resistant form of prion protein, termed PrP^{Sc}, appears to propagate itself in infected hosts by inducing the conversion of its normal host-encoded protease-sensitive precursor, PrP^{C}, into PrP^{Sc}. PrP^{C} is a monomeric glycophosphatidylinositol-linked glycoprotein that is low in β-sheet content, and highly protease-sensitive. Conversely, PrP^{Sc} aggregates are high in β-sheet content and partially protease-resistant.

As used herein “PMCA” or “Protein Misfolding Cyclic Amplification” refers to a method for amplifying PrP^{Sc} in a sample by mixing PrP^{C} with the sample, incubating the reaction mixture to permit PrP^{Sc} to initiate the conversion of PrP^{C} to aggregates comprising PrP^{Sc}, fragmenting any aggregates formed during the incubation step (usually by sonication), and repeating one or more cycles of the incubation and fragmentation steps. PMCA is based on the ability of prions to replicate in vitro in tissue homogenates containing PrP^{C} and is discussed in WO0204954, which is incorporated by reference herein. In some instances, incubation and sonication are alternated over a period of approximately three weeks, and intermittently a portion of the reaction mix is removed and incubated with additional PrP^{C} in order to serially amplify the PrP^{Sc} in the sample. Following the repeated incubation/sonication/dilution steps, the resulting PrP^{Sc} is detected in the reaction mix. PMCA has a number of limitations, notably the time required to achieve optimal sensitivity (about 3 weeks).

As used herein, the term “aggregate” includes aggregates, dimers, multimers, and polymers of prion proteins, for instance aggregates, dimers, multimers, and polymers of PrP^{Sc}.

As used herein, “agitation” includes introducing any type of turbulence or motion into a mixture or reaction mixture, for example, by sonication, stirring, or shaking, such as vortexing. In some embodiments, agitation includes the use of force sufficient to fragment PrP^{Sc} aggregates, which disperses PrP^{Sc} aggregates to facilitate further amplification. In some embodiments, fragmentation includes complete fragmentation, whereas in other embodiments, fragmentation is only partial.

In some embodiments, the invention provides a method for amplifying and detecting PrP^{Sc} in a sample. The method includes i) contacting the sample with a source of PrP^{C} to make a reaction mixture; (ii) incubating the reaction mixture; (iii) agitating the reaction mixture of (ii) in the presence of one or more beads; and (iv) detecting the amplified PrP^{Sc}. In some embodiments, steps (ii) and (iii) are repeated one or more times before step (iv) is conducted. In step (ii) of the reaction, aggregation of the PrP^{C} with the PrP^{Sc} can result in a conversion of the PrP^{C} to PrP^{Sc}.

In some examples, steps (ii) and (iii) are repeated (incubation/agitation cycles) from about 1 to about 500 times, from about 5 to about 300 times, or from about 10 to about 100 times. In some embodiments, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or more incubation/agitation cycles are performed.

In some embodiments, a portion of the reaction mixture can be diluted and added to additional PrP^{C} after a number of incubation/agitation cycles have been completed (e.g., “a round”), and then subjected to one or more further rounds of incubation/agitation cycles. In some embodiments, one additional round is performed. In other embodiments, 2, 3, 4, 5, 6, 7, 8, 9, or 10 additional rounds are performed to amplify the PrP^{Sc}.

In some embodiments, the reaction mixture is diluted about 2-fold between one or more rounds. In some embodiments, the reaction mixture is diluted about 4-fold, about 8-fold, about 10-fold, about 15-fold, about 25-fold, about 50-fold, about 100-fold, about 500-fold, about 1000-fold, about 5000-fold, about 10,000-fold, about 50,000-fold, or about 100,000-fold or more between rounds.

The period of time for incubation between agitations can vary and is not limiting. In some embodiments, the incubation time is approximately the same during each incubation/agitation cycle. In other embodiments, the incubation time is not the same during each incubation/agitation cycle. In some embodiments, in the first cycle, the period of time for incubation before the first agitation can be shorter, in some cases, much shorter, than the incubation time in one or more successive cycles. In some embodiments, the period of time for incubation is approximately 5 minutes, approximately 10 minutes, approximately 15 minutes, approximately 20 minutes, approximately 25 minutes, approximately 30 minutes, approximately 35 minutes, approximately 40 minutes, approximately 45 minutes, approximately 50 minutes, approximately 55 minutes, approximately 60 minutes, approximately 75 minutes, approximately 90 minutes, approximately 120 minutes or longer.

The period of time for agitation can vary and is not limiting. In some embodiments, the reaction mixture is agitated for approximately the same amount of time in each cycle. In other embodiments, the reaction mixture is agitated for a period of time that is not the same in each cycle. In some embodiments, the reaction mixture is agitated by sonication. In some embodiments, the reaction mixture is sonicated for approximately 5 seconds, approximately 10 seconds, approximately 15 seconds, approximately 20 seconds, approximately 25 seconds, approximately 30 seconds, approximately 35 seconds, approximately 40 seconds, approximately 45 seconds, approximately 50 seconds, approximately 55 seconds, approximately 60 seconds, approximately 75 seconds, approximately 90 seconds, approximately 120 seconds, approximately 150 seconds, approximately 180 seconds or longer. The sonication can be continuous or pulsed. In some embodiments, the reaction mixture is continuously sonicated, and not pulsed during the agitation step. In some embodiments, agitating the reaction mixture includes shaking or vortexing, for example, for about 1-180 seconds, or in some embodiments, for about 10 to 60 seconds. In some embodiments, the sonication energy is controlled, for example, by choosing % of power efficiency. In some embodiments, sonication pulses are delivered at 50% to 70% of power efficiency. In some embodiments, the sonication % power efficiency can be between 0% and 100%. In some embodiments, the % power efficiency is about 10%, about 20%, about 30%, about 40%, about 50%, about 60%, about 70%, about 80%, about 90%, or about 100%. In some embodiments, a Misonix-4000 sonicator is used to carry out the methods of the invention.

In some embodiments, the reaction mixture is incubated at about 25 to 70° C., and in some embodiments, the reaction mixture is incubated at about 37 to 55° C., or 45 to 55° C. In some embodiments, the reaction mixture is incubated at about 37° C.

In some embodiments, the incubation is performed under approximate physiological conditions (e.g., pH, temperature, and ionic strength). In some embodiments, protease inhibitors and one or more detergents are also added to the solution. The conditions will be chosen to permit conversion of PrP^{C} to PrP^{Sc}. In some embodiments, the incubation is performed in Ca²⁺-free and Mg²⁺-free PBS, pH 7.5, supplemented with 0.15 M NaCl, 1.0% Triton and protease inhibitors (e.g., 1 tablet of Complete protease inhibitors cocktail (Roche, Cat. #1836145) per 50 ml of conversion buffer).

The total reaction time for PMCAb can vary. In some embodiments, the total reaction time including agitation and incubation can be about 1 to about 160 hours or longer, such as about 2, about 4, about 6, about 8, about 16, about 20, about 24, about 36, about 42, about 48 hours, about 56 hours, about 72 hours, about 80 hours, about 88 hours, or about 96 hours or longer.

In some embodiments of PMCAb, the method includes incubating the reaction mixture for approximately 30-60 minutes; and sonicating the reaction mixture for approximately 30 seconds. In some embodiments, the incubation and sonication steps are repeated for approximately 1-160 hours. In some embodiments, three rounds of 48 cycles each are performed, and the samples are diluted 10-fold at the start of the new round. In some embodiments, the incubation and sonication steps are repeated for about 4 hours, about 8 hours, about 12 hours, about 18 hours, about 24 hours, about 30 hours, about 36 hours, about 40 hours, about 44 hours, about 48 hours, about 52 hours, about 56 hours, about 60 hours, about 64 hours, about 68 hours, about 72 hours, about 76 hours, about 80 hours, about 84 hours, about 88 hours, about 92 hours, about 96 hours or longer.

In accordance with the invention, the reaction mixture containing the source of PrP^{C} and test sample is agitated in the presence of one or more beads. The type of beads that can be used in the reaction is not limiting. In some embodiments, the one or more beads are made from polymeric substances. In some embodiments, the beads are made from polytetrafluoroethylene (PTFE; TEFLON), stainless steel, neoprene, nylon, ethylene propylene diene monomer (EPDM), nitrile rubber, zytel nylon, acetal, glass, ceramic, polypropylene or polystyrene. In some embodiments, the one or more beads can be obtained from commercial sources (e.g., Small Parts, Inc.) and can include PTFE Ball Grade II (Teflon), Stainless Steel 440C Ball Grade 24, Neoprene Ball, Nylon Ball, EPDM Ball, Nitrile Rubber Ball, Stainless Steel 302 Ball Grade 100, Metal Ball Grade I, and combinations thereof.

In some embodiments, the beads have a diameter of at least about 0.1 mm, at least about 0.2 mm, at least about 0.3 mm, at least about 0.4 mm, at least about 0.5 mm, at least about 0.6 mm, at least about 0.7 mm, at least about 0.8 mm, at least about 0.9 mm, at least about 1 mm, at least about 1.1 mm, at least about 1.2 mm, at least about 1.3 mm, at least about 1.4 mm, at least about 1.5 mm, at least about 1.6 mm, at least about 1.7 mm, at least about 1.8 mm, at least about 1.9 mm, at least about 2.0 mm, at least about 2.1 mm, at least about 2.2 mm, at least about 2.3 mm, at least about 2.4 mm, at least about 2.5 mm, at least about 2.6 mm, at least about 2.7 mm, at least about 2.8 mm, at least about 2.9 mm, or at least about 3.0 mm. In some embodiments, the beads have a diameter that is larger than 3.0 mm, such as beads having a diameter of about 3.5 mm, about 4.0 mm, about 4.5 mm, about 5.0 mm or larger. In some embodiments, the beads have a diameter of about 1.59 mm or about 2.38 mm.

Agitation can be conducted in the presence of any number of beads, and includes one or more beads, such as one bead, 2 beads, 3 beads, 4 beads, 5 beads, 6 beads, 7 beads, 8 beads, 9 beads, 10 beads, 11 beads, 12 beads, 13 beads, 14 beads, 15 beads, 16 beads, 17 beads, 18 beads, 19 beads, 20 beads, 21 beads, 22 beads, 23 beads, 24 beads, 25 beads, or more. In some embodiments, about 3.5 beads are used in the reaction. In some embodiments, the number of beads is greater than 25. For example, 30 beads, 40 beads, 50 beads, 60 beads, 70 beads, 80 beads, 90 beads, 100 beads, 150 beads, 200 beads, 250 beads or more can be used. In some embodiments, more than 25 beads are employed when the size of the bead is small, for example, 0.9 mm or less.

In some embodiments, the volume occupied by the one or more beads in the reaction mixture is about 5% of the total volume. In some embodiments, the beads occupy about 10%, about 15%, about 20%, about 25%, about 30%, about 35%, about 40%, about 45%, about 50%, about 55%, about 60%, about 65%, about 70%, or about 75% or more of the reaction mixture volume. In some embodiments, the beads used in a reaction mixture (when more than one is present) are all of the same type and size. In other embodiments, the reaction mixture contains mixtures of beads of varying sizes and/or types.

In some embodiments, the beads are also present during one or more incubation steps of the method. In some embodiments, the beads are present throughout the incubation and agitation steps. In some embodiments, the beads can also be present during the detection step.

The amplification fold of the PrP^{Sc} in PMCAb is significantly greater than the amplification observed in the absence of the beads. In some embodiments, within a single round, the amplification fold of PMCAb is increased relative to standard PMCA (without beads) at least about 10-fold to at least about 1000-fold. In some embodiments, the increase is at least about 50-fold to at least about 1000-fold, or at least about 75-fold to at least about 635-fold, depending on the initial dilution of the test sample. In some embodiments, the amplification fold is at least about 10-fold, about 25-fold, about 50-fold, about 75-fold, about 150-fold, about 300-fold, about 450-fold or about 635-fold. In some embodiments, the fold amplification is greater than 635-fold, including about 700-fold, about 800-fold, about 900-fold, or about 1000-fold.

In some embodiments, the yield of conversion of PrP^{C} to PrP^{Sc} is greater than 50%. In some embodiments, the yield of conversion is greater than 60%, 70%, 80%, 90%, 95%, 96%, 97%, 98% or 99%. In some embodiments, the yield of conversion approaches 100% or even achieves 100%.

Prion Diseases

In some embodiments, the methods are used to diagnose a prion disease. In some embodiments, the prion disease can include, but is not limited to scrapie (typical and atypical forms) in sheep, bovine spongiform encephalopathy (BSE; also known as mad cow disease, including classical and the atypical forms BSE-H and BSE-L) in cows, bovine amyloidotic spongiform encephalopathy (BASE) in cows, transmissible mink encephalopathy (TME) in mink, chronic wasting disease (CWD) in elk, moose, or deer, feline spongiform encephalopathy, ungulate encephalopathy in nyala, oryx and greater kudu, and Creutzfeldt-Jakob disease (CJD) and its varieties (including but not limited to iatrogenic Creutzfeldt-Jakob disease (iCJD), variant Creutzfeldt-Jakob disease (vCJD), genetic Creutzfeldt-Jakob disease (fCJD), and sporadic Creutzfeldt-Jakob disease (sCJD)), Gerstmann-Straussler-Scheinker syndrome (GSS), fatal familial insomnia (fFI), sporadic fatal insomnia (sFI), kuru, and Alpers syndrome in humans. Reviews regarding various prion diseases can be found in the following documents, which disclosures relating to the prions and their diseases are incorporated by reference herein: Prusiner S B., N Engl J. Med., 17:344(20):1516-26 (2001); Tranulis M A, Benestad S L, Baron T, Kretzschmar H., Atypical Priori Diseases in Humans and Animals, Top Curr Chem. 2011 May 20. [Epub ahead of print]; Cali et al. Brain. September; 129 (Pt 9):2266-77 (2006); Capellari et al. Acta Neuropathol. 121(1)11-37 (2011) Epub 2010, Oct. 27.

In some embodiments, the sample comes from a strain known to harbor PrP^{Sc}. In some embodiments, the prion sample is derived from sheep, goat, or mink. In some embodiments, the prion is from sheep, goat, or mink and has been propagated and stabilized in rodent models, such as hamster or mouse. In some embodiments the rodent model is a hamster stain, and can include strain 263K (Sc237), Drowsy, Hyper, 139H, ME7 or SSLOW. In some embodiments, the rodent model is a mouse stain, and can include strain RML or 22L.

In some embodiments, the method enables the rapid testing of live animals for infection to protect against unnecessary culling of herds or inadvertent introduction of prions into the food chain. The disclosed methods can offer advantages over available methods for diagnosis of these neurologic disorders. For instance, conventional PMCA takes up to three weeks to perform, whereas the disclosed methods provide that a positive diagnosis can be made in as little as 1 day. In some embodiments, one or more samples are obtained from peripheral tissues, such as blood and cerebral spinal fluid (CSF). In some embodiments, the methods provide sensitivity that is sufficiently high to detect or diagnose disease prior to the onset of clinical symptoms.

In some embodiments, PrP^{Sc} includes mammalian prion protein, and in some embodiments, the source of PrP^{C} includes a detectable label, enabling rapid detection of PrP^{Sc}.

Sources of PrP^{C}

In accordance with the invention, the source of PrP^{C} is not limiting. In some embodiments, the source of the PrP^{C} is from the same species as the source of the test sample. In some embodiments, a mixture of sources of PrP^{C} is used. In some embodiments, the PrP^{C} source is of human, bovine, ovine, hamster, rat, mouse, canine, feline, goat, cervid, or non-human primate origin. The source can include naturally occurring sources, such as tissue or fluid samples from normal animals, which can include purified, unpurified or homogenized samples, cultured cells which express PrP^{C} or isolates from the cells, or from sources wherein the PrP^{C} has been recombinantly or synthetically produced.

In some embodiments, the source is normal brain or central nervous system tissue, or homogenates or fractions thereof, in some embodiments, the source is normal brain homogenate or a fraction thereof from human, bovine, ovine, hamster, rat, mouse, canine, feline, cervid or non-human primate origin.

In some embodiments, the source is from cultured cells, which can include human or non-human cells which express PrP^{C}. In some embodiments, the cultured cells are human cells, bovine cells, goat cells, ovine cells, hamster cells, rat cells, mouse cells, canine cells, feline cells, cervid cells or cells of non-human primate origin. In some embodiments, the PrP^{C} source can be primary cell cultures or cells derived from established cell lines. In some embodiments, the cultured cells are HeLa cells.

In some embodiments, PrP^{C} is recombinantly produced. Recombinant sources can also include variant PrP^{C} sequences. Various recombinant sources are discussed in U.S. Patent Application Publication No.: 2009/0047696 A1, which disclosure is incorporated herein by reference in its entirety. Examples of recombinant prion proteins that can be used include, for example: Homo sapiens (Genbank Accession No: BAA00011), Xenopus laevis (Genbank Accession No: NP001082180), Bos Taurus (Genbank Accession No: CAA39368), Dania verio (Genbank Accession No: NP991149), Tragelaphus strepsiceros (Genbank Accession No: CAA52781), Ovis aries (Genbank Accession No: CAA04236), Trachernys scripta (Genbank Accession No: CAB81568), Gallus gallus (Genbank Accession No: AAC28970), Rattus norvegicus NP036763), Mus musculus (Genbank Accession No: NP035300), Monodelphis domestica (Genbank Accession No: NP001035117), Giraffa camelopardalis (Genbank Accession No: AAD13290), Oryctolagus cuniculus (Genbank Accession No: NP001075490), Macaca mulatto (Genbank Accession No: NP001040617), Bubalus bubalus (Genbank Accession No: AAV30514), Tragelaphus imberbis (Genbank Accession No: AAV30511), Boselaphus tragocamelus (Genbank Accession No: AAV30507), Bos garus (Genbank Accession No: AAV 30505), Bison bison (Genbank Accession No: AAV30503), Bos javanicus (Genbank Accession No: AAV30498), Syncerus coffer coffer (Genbank Accession No: AAV30492), Syncerus coffer nanus (Genbank Accession No: AAV30491), and Bos indicus (Genbank Accession No: AAV30489).

In some embodiments, host cells are transformed with a nucleic acid that encodes PrP^{C}, fragments or variants thereof. Host cells can include, but are not limited to mammalian cells, bacterial cells, yeast cells, insect cells, whole organisms, such as transgenic mice, or other cells that can serve as source of the PrP^{C}. In some embodiments, the cell is a bacterial cell, such as an E. coli cell. In some embodiments, crude cell lysates or purified PrP^{C} from PrP^{C} expressing cells can be used as the source of the PrP^{C}. In some embodiments, the recombinant PrP^{C} can be fused to an amino acid sequence to facilitate expression and/or purification of the recombinant protein. In some embodiments, the source of PrP^{C} is a transgenic animal that expresses PrP^{C}. Use of lysates for PMCA from cultured cells transformed with PrP^{C} is discussed in Mays et al. PLoS One, 6(3):e18047 (2011) and Yokoyama et al. Neuroscience Letters, 498:119-123 (2011). Use of transgenic animals as a source for PrP^{C} for PMCA has been discussed in Green et al. PLoS Pathogens, 4(8):e1000139 (2008). In some embodiments, a source of PrP^{C} is a transgenic mouse expressing human PrP^{C}.

For example, full length recombinant PrP^{C}, fragments of recombinant PrP^{C}, as well as variants of the wild type sequence wherein one or more amino acids has been substituted, deleted or inserted, or combinations thereof, can be used in accordance with the methods of the invention. Chimeric recombinant PrP^{C} can also be used, wherein a portion of the protein is from one species, and a portion of the protein is from another species. In some embodiments, a functional fragment of PrP^{C} can aggregate with PrP^{Sc} and result in a conversion of the PrP^{C} to PrP^{Sc}.

In some embodiments, the reaction is carried out under conditions that will minimize the spontaneous conversion of recombinant PrP^{C} into PrP^{Sc}, such as the conditions disclosed in U.S. Appl. Pub. No.: 2009/0047696, which is incorporated herein by reference. In some embodiments, the reaction conditions include the use of a detergent, such as both an ionic and a non-ionic detergent. For example, the conditions can include the combination of about 0.05-0.1% of an ionic detergent such as SDS and about 0.05-0.1% of a nonionic detergent such as TX-100 in the reaction mixture. Other conditions can include the use of shaking instead of sonication, and the use of cycles of shaking/rest that are about 1:1 in duration. In some embodiments, the reactions can be carried out at about 37-60° C., for example about 45-55° C.

Test Sample

The sample to be amplified or tested in accordance with the invention can include any sample capable of harboring PrP^{Sc}. In some embodiments, the sample is a tissue sample (or part thereof or in homogenized form) or other sample of bodily origin including, but not limited to, blood, lymph nodes, brain tissue (includes whole brain, anatomical parts, or fractions thereof), spinal cord, tonsils, internal organs (such as spleen, stomach, pancreas, liver, intestine (large or small), lungs, heart, thymus, bladder or kidney, for example), skin, muscle, appendix, olfactory epithelium, nasal tissue, cerebral spinal fluid, urine, feces, milk, mucosal secretions, tears and/or saliva. Other compositions from which samples can be taken include food stuffs (either for human or animal consumption), drinking water, forensic evidence, surgical instruments, and/or mechanical or medical devices. In some embodiments, environmental samples are tested for the presence of PrP^{Sc}. In some embodiments, environmental samples, such as soil, water, or plants can be sampled and tested for the presence of PrP^{Sc}. For example, deer can be infected with prions and the soil or other parts of the environment in deer habitats can be contaminated with prions and it could be a health hazard for humans who might become infected with the prions.

In some embodiments, the methods of the invention can be used to test blood or blood components, such as human blood or blood components for medical use to ensure safety. In some embodiments, the blood or blood components to be tested include whole blood, plasma, serum, red blood cells, white blood cells, platelets or Factor VIII. In some embodiments, the blood or blood components to be tested is of non-human origin. In some embodiments, blood or blood components are tested from non-human animals.

In some embodiments, the methods of the invention can be used to amplify and detect prions in cosmetics, biopharmaceuticals, pharmaceuticals, or reagents used in the development of such products, including for research and development purposes. In some embodiments, the component to be tested is animal serum which is used in cell culture, such as fetal bovine serum.

In some embodiments, agricultural animals can be tested, including animals that are used to produce meat and/or milk, such as cows, goat, sheep and the like. In some embodiments, a tissue sample or other sample of bodily origin is obtained from the animal and tested in accordance with the methods of the invention. In some embodiments, the product intended for consumption is tested, such as the meat, milk, yoghurt, cream, or cheese.

Detection of PrP^{Sc} Following Amplification

In some embodiments, PrP^{Sc} is detected in the reaction mixture following the amplification steps. PrP^{Sc} can be detected following amplification using a variety approaches and the method for detecting PrP^{Sc} is not limiting. Direct and indirect methods can be used for detection of PrP^{Sc}. In some embodiments, PrP^{Sc} is separated from remaining PrP^{C} in the reaction mixture. In some embodiments, the PrP^{C} is removed by protease treatment. In some embodiments, reaction mixtures are incubated with Proteinase K (PK), for example, at a concentration of 1-50 μg/ml of PK for about 1 hour at 37° C. In some embodiments, the reactions with PK can be stopped prior to assessment of prion levels by addition of PMSF or electrophoresis sample buffer.

In some embodiments, the PrP^{Sc} can also be separated from PrP^{C} by use of ligands that specifically bind PrP^{Sc}, including conformational antibodies, certain nucleic acids, plasminogen, PTA and/or various peptide fragments.

In some embodiments, PrP^{Sc} is detected using a Western blot, an ELISA assay, a CDI assay, a DELPHIA assay, radioimmunoassay, a strip immuno chromatographic assay, a spectroscopic assay, a fluorescence assay, a radiometric assay or some combination of these approaches. In some embodiments, the ELISA assay is a two-site immuno metric sandwich ELISA. Exemplary antibodies for detection of PrP^{Sc} can include the 3F4 monoclonal antibody, monoclonal antibody D13 (directed against residues 96-106 (Peretz et al. Nature 412, 739-743 (2001)), polyclonal antibodies R18 (directed against residues 142-154), and R20 (directed against C-terminal residues 218-232) (Caughey et al. J. Virol. 65, 6597-6603 (1991)).

In some embodiments, PrP^{Sc} is detected by bioassay by virtue of its ability to cause prion disease or symptoms in an animal. Animals can be inoculated with the reaction mixture or a dilution thereof, and assayed for the presence of prion disease characteristics or symptoms, including behavioral, biochemical, histochemical, and/or anatomical features thereof. In some embodiments, the reaction mixture is diluted about 10-fold, about 10²-fold, about 10³-fold, about 10⁴-fold, about 10⁵-fold, about 10⁶-fold, or about 10⁷-fold prior to inoculation of the animal. Taking into account the dilution of the starting test sample, and dilutions during various rounds of PMCAb, the final dilution of the test sample administered in a bioassay, in some embodiments is about 10⁶-fold, about 10⁷-fold, about 10³-fold, about 10⁹-fold, about 10¹⁰-fold, about 10¹¹-fold, about 10¹²-fold, about 10¹³-fold, about 10¹⁴-fold or more.

In some embodiments, PrP^{Sc} concentration is estimated by Western blot followed by densitometric analysis, and comparison to Western blots of samples for which the concentration of prion protein is known. For example, this can be accomplished by scanning data into a computer followed by analysis with quantitation software. In some embodiments, several different dilutions of the sample generally are analyzed in the same gel.

Screening Methods

In some embodiments, the methods of the invention can be used to identify agents that modify the ability of prions to replicate, such as agents that would be candidates for the treatment of prion diseases. As used herein, the term “agent” refers to any synthetically produced or naturally occurring molecule that potentially can inhibit or enhance prion function activity. The candidate agent can include, without limitation, a protein or fragment thereof, a small molecule, a polymer or nucleic acid molecule. In some embodiments, candidate agents can include fragments or parts of naturally-occurring compounds, or can be found as active combinations of known compounds, which are otherwise inactive. Compounds isolated from natural sources, such as animals, bacteria, fungi, plant sources, including leaves and hark, and marine samples, can be assayed as candidates for the presence of potentially useful agents.

In some embodiments, the invention provides a method of screening to identify agents that modulate PrP^{Sc} formation, comprising i) contacting a sample having PrP^{Sc} with a source of PrP^{C} to make a reaction mixture; (ii) incubating the reaction mixture in the presence and absence of the agent; (iii) agitating the reaction mixture of (ii) in the presence of one or more beads; and (iv) detecting the amplified PrP^{Sc} generated in the presence and absence of the agent. In some embodiments, steps (ii) and (iii) are repeated one or more times before step (iv) is conducted. When a difference between the levels of PrP^{Sc} in samples is detected, agents are identified that either enhance or inhibit conversion of PrP^{C} to PrP^{Sc}.

Using this method, screening of large libraries of candidate agents can be conducted, or specific classes of agents or specific agents can be individually tested. In some embodiments, multiple agents are pooled in a single reaction and tested for their ability to modify conversion of PrP^{C} to PrP^{Sc}. If a positive result is obtained, the active agent can be subsequently identified from the candidate pool.

Kits

The invention further provides kits for amplifying and/or detecting a prion in a sample that includes a source of PrP^{C} and one or more beads. In some embodiments, a kit can include, in suitable container, one or more of the following: 1) a reaction mixture buffer; 2) decontamination solution; 3) a positive control sample containing PrP^{Sc}; 4) a negative control sample that does not contain PrP^{Sc}; 5) a source of PrP^{C}; 6) one or more beads; 7) one or more proteases, such as proteinase K; and 8) one or more reagents for the detection of PrP^{Sc}. In some embodiments, the reagent for detection of PrP^{Sc} can include one or more antibodies.

In some embodiments, the kit includes a reaction mixture buffer. In some embodiments, the reaction mixture buffer includes one or more detergents, such as SDS and TX-100. In some embodiments, the source of PrP^{C} is provided in liquid form. In some embodiments, it is provided in lyophilized form. In some embodiments, the source of PrP^{C} is provided as a tissue sample. In some embodiments, the tissue sample has been processed, for example, fractionated or homogenized.

Any of the compositions described herein can be included in a kit for carrying out PMCAb as described herein. If a recombinant PrP^{C} is used as a source, the kit can further include reagents for expressing or purifying a recombinant PrP^{C}. In some embodiments, the kit also can include pre-labeled PrP or reagents that can be used to label the PrP^{C}, with for example, radioisotopes or fluorophores.

Regents for the detection of prions can also include pre-coated microtiter plates for ELISA and/or conformation-dependent immunoassay (CDI) detection of PrP^{Sc} and antibodies for use in ELISA, CDI, strip immunochromatography or Western blot detection methods.

### EXAMPLES

### Example 1

### Highly Efficient Protein Misfolding Cyclic Amplification

### Materials and Methods

Protein Misfolding Cyclic Amplification

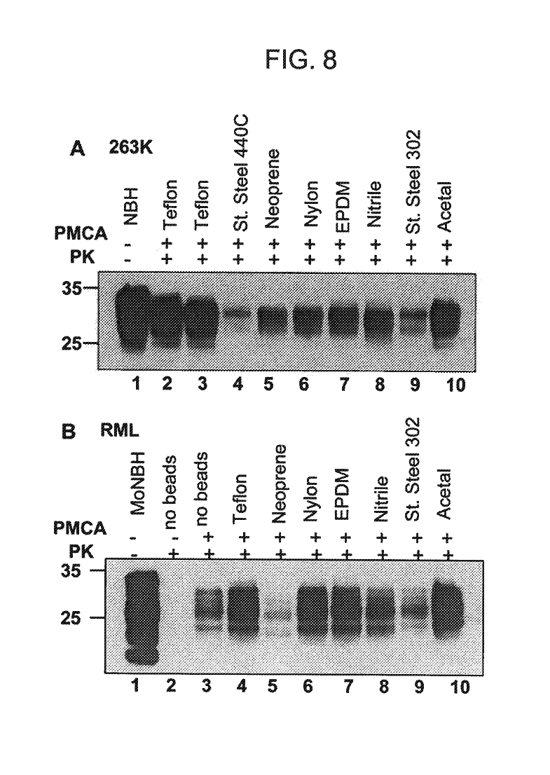

Healthy hamsters were euthanized and immediately perfused with PBS, pH 7.4, supplemented with 5 mM EDTA. Brains were dissected, and 10% brain homogenate (w/v) was prepared using ice-cold conversion buffer and glass/Teflon tissue grinders cooled on ice and attached to a constant torque homogenizer (Heidolph RZR2020). The brains were ground at low speed until homogeneous, then 5 additional strokes completed the homogenization. The composition of conversion buffer was as previously described (Castilla et al. cell 121: 195-206 (2005)); Ca²⁺-free and Mg²⁺-free PBS, pH 7.5, supplemented with 0.15 M NaCl, 1.0% Triton and 1 tablet of Complete protease inhibitors cocktail (Roche, Cat. #1836145) per 50 ml of conversion buffer. The resulting 10% normal brain homogenate in conversion buffer was used as the substrate in PMCA reactions. To prepare seeds, 10% scrapie brain homogenates in PBS were serially diluted 10- to 10¹⁴-fold, as indicated, in the conversion buffer and 10 μl of the dilution were used to seed 90 μl of NBH in PMCA. Samples in 0.2 ml thin-wall PCR tubes (Fisher, Cat. #14230205) were placed in a rack fixed inside Misonix S-3000 or S-4000 microplate horn, filled with 300 ml water. Two coils of rubber tubing attached to a circulating water bath were installed for maintaining 37° C. inside the sonicator chamber. The standard sonication program consisted of 30 sec sonication pulses delivered at 50% to 70% efficiency applied every 30 min during a 24 hour period. For PMCA with beads, small (1.58 mm diameter) or large (2.38 mm diameter) Teflon beads (McMaster-Carr, Los Angeles, Calif.) were placed into the 0.2 ml tubes first using tweezers, then NBH and seeds were added. The following beads from Small Parts, Inc. were tested in FIG. 8: PTFE Ball Grade II (Teflon); Stainless Steel 440C Ball Grade 24; Neoprene Ball; Nylon Ball; EPDM Ball; Nitrile Rubber Ball; Stainless Steel 302 Ball Grade 100; Acetal Ball Grade I. The diameter of all beads was 2.38 mm except of Stainless Steel 440C Ball, which was 2 mm in diameter. The following low binding beads showed no effects on efficiency in PMCA: Silica Beads Low Binding 800 or 400 μm diameter, and Zirconium Beads Low Binding 200 or 100 μm diameter (all from OPS Diagnostics LLS, Lebanon, N.J.).

In our experience, the amplification efficiency in PMCA depended strongly on the position of the tube within a microplate horn, i.e. distance of a tube from horn's surface to the tube and its center; and the age of the sonicator's horn. In the current studies, several Misonix sonicators were used, all equipped with horns less than one year old. The tubes were placed only in positions between 1.5 cm and 5 cm from the horn's center. Nevertheless, we experienced substantial variations in amplification efficiency in standard PMCA (no beads), which appear due to differences in the age of horns, individual patterns of horn corrosion or differences in the horizontal coordinates of tubes. In the presence of beads, the amplification was much more robust and showed only minor variations.

Bioassay and Estimation of the Incubation Times to Disease

Weanling golden Syrian hamsters were inoculated intracerebrally with 50 μl each using the following inocula: animals of group 1 were inoculated with 263K brain homogenate diluted 10⁴-fold relative to whole brain in PBS, 1% BSA. For groups 2 and 3, 10 μl of 10% 263K scrapie brain homogenate were mixed with 90 μl of PBS and subjected to a sonication procedure equivalent to a single PMCA round (48 sonication cycles) in the absence of NBH. Sonication was performed either without beads (for group 2) or with 3 large beads (for group 3). Then, the sonication products were diluted 1.00 fold into PBS, 1% BSA to obtain final dilution of 10⁻⁴ relative to whole 263K brain for inoculation. For groups 4 and 5, PMCA reactions were seeded with 10⁻⁴ diluted 263K brain material, then six serial PMCA rounds were conducted in the absence of beads (for group 4) or presence of 3 large beads (for group 5) using 1:10 dilutions between rounds. After the 6^{th} round of serial PMCA, the amplification products were diluted an additional 10-fold into PBS, 1% BSA to obtain final 10¹⁰-fold dilutions of the initial 263K brain material prior to inoculation.

In hamsters inoculated with the 263K scrapie strain, the asymptomatic period of infection lasts 60 to 160 days followed by a stereotypic clinical progression leading to death 2 to 3 weeks later. Individual symptoms, such as wobbling gait and head bobbing, are readily recognized but their onset is subtle and subject to large inter (and even intra) observer variability. Incubation time determinations are greatly improved by an empirical determination of endpoint (Gregori et al. Transfusion 46: 1152-1161 (2006)). The adult body weight of asymptomatic or uninfected hamsters is stable or increases slowly during adulthood but drops precipitously during symptomatic disease. Hamsters showing clear signs of early scrapie were individually caged and weighed daily. The weight history was plotted against time with a reference endpoint line marking 20% of the maximum weight registered. Animals were euthanized when their body weights dropped below 20% of maximum body weight and the incubation endpoint was taken as the time intercept of the 20% line.

Proteinase K Assay

To analyze production of PK-resistant PrP material in PMCA, 15 of each sample were supplemented with 2.5 μl SDS and 2.5 μl PK, to a final concentration of SDS and PK of 0.25% and 50 μg/ml respectively, followed by incubation at 37° C. for 1 hour. The digestion was terminated by addition of SDS-sample buffer and boiling for 10 min. Samples were loaded onto NuPAGE 12% BisTris gels, transferred to PVDF membrane, and stained with 3F4 or D18 antibody for detecting hamster or mouse PrPs, respectively.

To analyze scrapie brain homogenates, an aliquot of 10% brain homogenate was mixed with an equal volume of 4% sarcosyl in PBS, supplemented with 50 mM Tris, pH 7.5, and digested with 20 μg/ml PK for 30 min at 37° C. with 1000 rpm shaking (Eppendorf themiomixer). The reaction was stopped by adding 2 mM PMSF and SDS sample buffer. Samples were boiled for 10 min and loaded onto NuPAGE 12% BisTris gels. After transfer to PVDF membrane, PrP was detected with 3F4 antibody.

Quantification of PrP^{Sc} by Dot Blotting

To obtain calibration curves for calculating of PMCA fold amplification, 10% brain homogenate from 263K animals was sonicated for 1 min and serially diluted into 10% NBH sonicated for 30 sec. PMCA samples as well as 263K dilutions were digested with 50 μg/ml PK for 1 h at 37° C. The reaction was stopped by addition of 2 mM PMSF. All samples were diluted 10-fold in PBS, and analyzed using a 96-well immunoassay similar to those previously published (Kramer et al. Prion 3:44-48 (2009)). Our procedure employed the Bio-Dot microfiltration system (Bio-Rad, Hercules, Calif.) used according to the instruction manual. 50 μl of diluted samples were loaded into each well and allowed to bind to a 0.45 μm Trans-Blot nitrocellulose membrane (Bio-Rad, Hercules, Calif.), Following two washes with PBS, the membrane was removed, incubated for 30 min in 6 M GdnHCl to enable PrP denaturation, washed, and probed with 3F4 antibody according to the standard immunoblotting procedure. Chemiluminescent signal from the membrane was collected with a Typhoon 9200 Variable Mode Imager (Amersham Biosciences, Piscataway, N.J.) and quantified with ImageQuant software (Amersham Biosciences).

Atomic Force Microscopy Imaging of rPrP-Fibrils

Full-length hamster rPrP (residues 23-231, no tags) was expressed and purified as previously described (Breydo et al. Biochemistry 44: 15534-15543 (2005); Ostapchenko et al. J Mol Biol 383: 1210-1224 (2008)). To prepare fibrils, the fibrillation reactions were conducted in 2M GdnHCl, 50 mM MES, pH 6.0 at 37° C. at slow agitation (˜60 rpm) and rPrP concentration of 0.25 mg/ml (Makarava et al. J Biol Chem 283: 15988-15996 (2008)). 0.2 ml PCR tubes containing 100 μl solution of rPrP fibrils (10 μg/ml) dialyzed into 5 mM sodium acetate, pH 5.0, were placed in a rack fixed on the top of Misonix S-4000 microplate horn filled with 300 ml water and sonicated in the absence or presence of five small Teflon beads at 200 W ultrasound power for 30 s. Then, 5 μl of each sample was placed on a freshly cleaved piece of mica, incubated for 5 min, washed gently with Milli Q water, dried on air, and analyzed using a Pico LE AFM system (Agilent Technologies, Chandler, Ariz.) equipped with a PPP-NCH probe (Nanosensors, Switzerland) and operated in tapping mode. Topography/amplitude images (square size of 2 to 10 μm, 512×512 pixels) were obtained at 1 line/s scanning speed. Tip diameter was calibrated by obtaining images of 5-nm gold particles (BBinternational, UK) under the same scanning conditions. Images were analyzed with PicoScan software supplied with the instrument. Particle height was calculated directly from the topography profiles, while width and length were measured at the half-height and corrected for the tip diameter. Dimensions of 130-150 particles for each group from three independent experiments were measured.

### Results

### Beads Significantly Improve the Yield and Rate of PMCA Reactions.

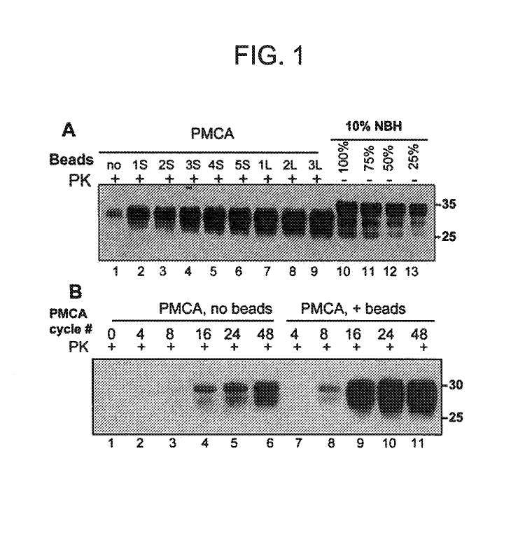

In the past, we found that beads with a diameters of 1.59 mm (referred to as small or S) or 2.38 mm (referred to as large or L) significantly accelerated the formation of amyloid fibrils of rPrP in vitro (Bocharova O V, Breydo L, Parfenov A S, Salnikov V V, Baskakov I V (2005) In vitro conversion of full length mammalian prion protein produces amyloid form with physical property of PrPSc. J Mol Biol 346: 645-659). Here, we tested whether beads have any effects on the rate of prion amplification in PMCA. In standard PMCA (sonication for 30 sec every 30 min, 48 cycles total, no beads), the typical yield of conversion of PrP^{C} into PrP^{Sc} was approximately 10% as judged by Western blotting (FIG. 1A). This amplification yield was consistent with previous studies on amplification of the 263K strain. In the presence of beads, however, the conversion yield improved significantly and approached 100% when 3 large or 5 small beads were used (FIG. 1A),

The kinetics of PrP^{Sc} amplification monitored by Western blotting revealed that in the absence of beads the newly generated PrP^{Sc} was detected by the 16^{th} cycle, whereas in the presence of beads it already was seen by the 8^{th} cycle (FIG. 1B). Furthermore, in the presence of beads the reaction reached a plateau in only 24 cycles and produced a much higher yield (FIG. 1B). These results illustrated that beads with diameters of 1.59 or 2.38 mm improved both the yield and the rate of 263K conversion. When beads of submillimeter diameter (800, 400, 200 or 100 μm, see Methods) were used instead, no noticeable increase in PrP^{Sc} amplification was observed (data not shown).

### PMCAb Amplifies Prion Infectivity.

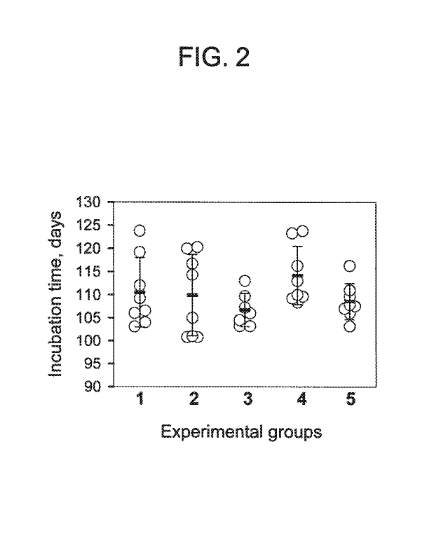

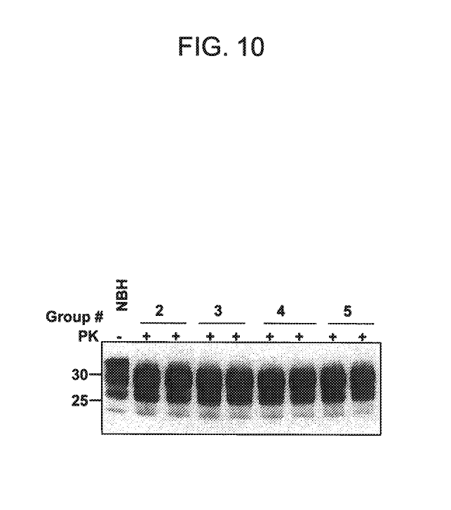

To test whether the products of PMCAb were infectious, the reactions were seeded with 10⁴-diluted 263K brain material and subjected to amplification in the presence or absence of beads for 6 rounds of 48 cycles each. The products of each round were diluted 10-fold into fresh NBH for the subsequent round. The PMCA products from the final round were then diluted an additional 10-fold prior to inoculation of 50 μl per animal. The final dilution of the initial 263K brain material was 10¹⁰ fold. In our laboratory the concentration of 263K scrapie in the brains of hamsters in the late stages of symptomatic disease is consistently between 1 and 2×10¹⁰ Infectious Dose₅₀/g of brain (Gregori et al. Transfusion 46: 1152-1161 (2006)). In the absence of amplification, a 10¹⁰ dilution of 263K brain would contain 1 ID₅₀/ml giving a probability of infection of 0.05 from a 50 μl inoculation. Nevertheless, all animals inoculated with PMCA products formed in the presence or absence of beads developed clinical disease with the mean value of endpoint 108.6±3.9 or 114.2±6.3 days post inoculation, respectively (FIG. 2, groups 4 and 5, respectively). Incubation time endpoints were determined empirically as described in the methods and as described. Id. The reference group inoculated with 10⁴-diluted 263K brain reached the endpoint by 110.5±7.5 days (FIG. 2, group 1). Bioassays of two 263K brain homogenates sonicated for 48 PMCA cycles (1 round) in the absence of a substrate revealed that sonication of PrP^{Sc} per se did not notably change its infectivity level regardless of whether beads were present or absent during the sonication cycles (FIG. 2, groups 2 and 3, respectively). Similar amounts of PrP^{Sc} were found in the brains from all animal groups (FIG. 10).

The bioassay experiment confirmed that prion infectivity is amplified in PMCAb. Without a titration experiment, it is difficult to establish accurate infectivity titers of PMCA or PMCAb products. Nevertheless, considering that group 5 gave the same incubation times as group 1, even though the amplification products were diluted an additional 10-fold prior to inoculation, the infectivity dose of PMCAb products appeared to be 10-fold higher than the dose in 10⁴-diluted 263K brain material.

### The Sensitivity of PrP Detection is Improved in PMCAb.

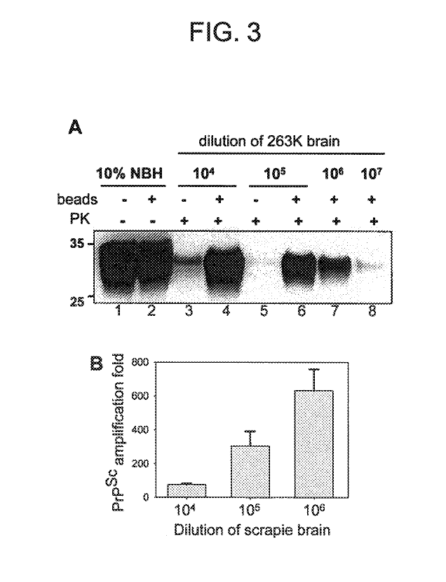

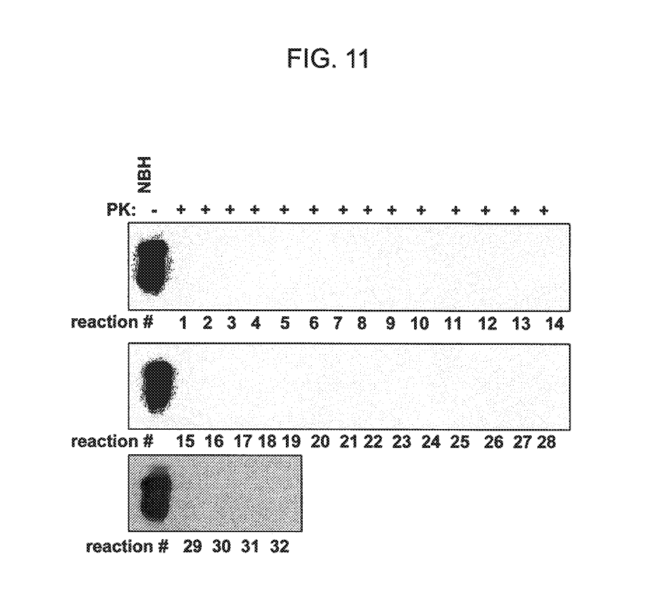

To test whether the application of beads improves the detection limit, serially diluted 263K brain homogenate was used to seed the PMCA reactions that consisted of 48 cycles. In the absence of beads, seeding with 10³-fold and with 10⁴-fold diluted scrapie brains gave sufficient amplification of PrP^{Sc} to be detected by Western blotting. In the presence of beads, however, the reactions seeded with 10-fold diluted 263K brains showed consistent, reproducible amplification for subsequent detection by Western blotting (FIG. 3). Frequently, sufficient amplification of PrP^{Sc} for detection by Western blotting was observed in the reactions with beads seeded with 10⁷-fold diluted scrapie brains. Therefore, within 48-cycle PMCA, beads improved the sensitivity of detection by at least 2 or 3 orders of magnitude. To rule out the possibility of the PrP^{Sc} formation de novo, 32 unseeded reactions were conducted, each of which consistent of 3 rounds of serial PMCAb (sPMCAb). None of them showed PK-resistant material on Western blotting (FIG. 11).

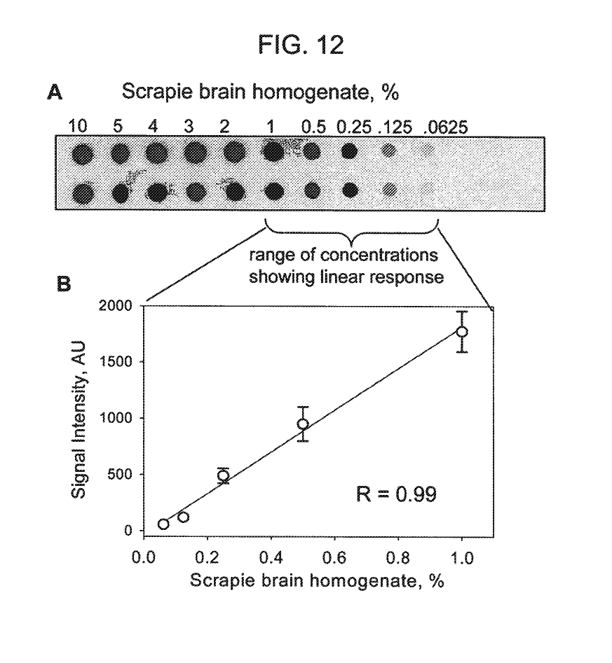

To estimate quantitatively the PrP^{Sc} amplification fold achieved in a single PMCAb round, we employed dot blotting as it provides a better linear response within a broader range of PrP^{Sc} concentrations than the Western blotting (FIG. 12). The PMCAb reactions seeded with 10⁴-, 10⁵-, or 10⁶-diluted 263K brain were found to produce reliable amplification by ˜75-, 300-, and 635-fold within 48 cycles, respectively (FIG. 3B). An increase in amplification fold at higher dilutions of seeds suggests that the effect of beads was most beneficial at high PrP^{C} to PrP^{Sc} ratios, where the reaction is no longer limited by the concentration of a substrate and/or cofactors.

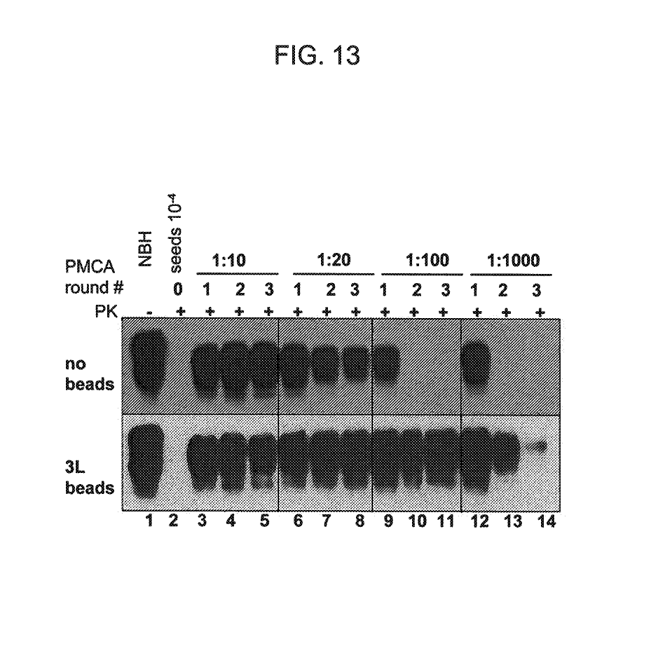

To estimate the PrP^{Sc} amplification fold using an alternative approach, sPMCA reactions consisted of three rounds were performed with the dilution factors between the rounds ranging from 1:10 to 1:1000. In the absence of beads, we observed a gradual decrease in the signal intensity as a function of PMCA round at dilutions of 1:20 indicating that the amplification fold in each round was slightly lower than 20 (FIG. 13). In the presence of beads, however, the signal was stable at 1:100 dilution but decayed at 1:1000 dilution, suggesting that the amplification fold in each round was higher than 100 but less than 1000 (FIG. 13). This experiment confirmed that up to several hundred fold amplification could be achieved in one PMCAb round consisted of 48 cycles, if the reaction is not limited by substrate and cofactors.

### Detection of Minute Amounts of PrP^{Sc}

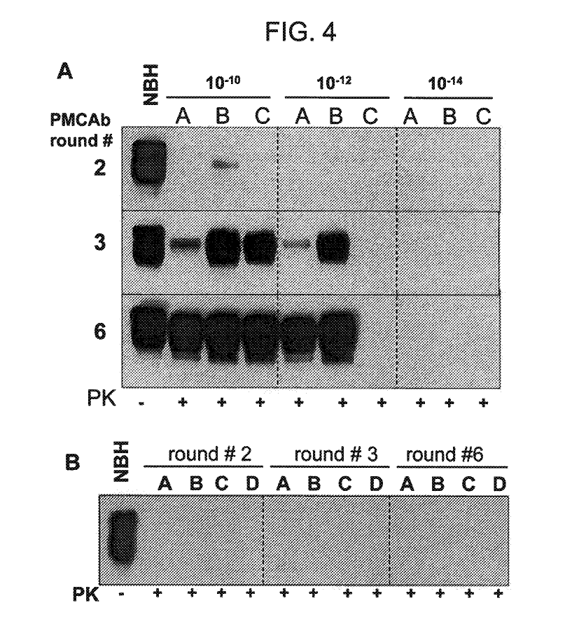

In previous studies, sPMCA of serially diluted 263K brain homogenate was used to determine the last dilution that still contained PrP^{Sc} particles (Saa et al, J Biol Chem 281: 35245-35252 (2006)). Three out of four reactions seeded with 10¹²-diluted 263K brain material were found to be positive, while five to seven sPMCA rounds, each consisting of 144 cycles, were required to amplify 10¹²-diluted 263K to levels detectible by Western blotting. Id. To test the effectiveness of PMCAb in amplifying minute quantities of PrP^{Sc}, 263K brain homogenate was serially diluted up to 10¹⁴-fold and then amplified in sPMCAb, where each round consisted of 48 cycles. 10¹²-diluted 263K brain material was detected in 4 out of 8 reactions in the third round (FIG. 4A). An increase in number of rounds to six did not increase the percentile of positive reactions seeded with 10¹²-diluted 263K brain nor did it reveal any positive signals in reactions seeded with 10¹⁴-diluted 263K brain (FIG. 4A). 10¹⁰-diluted 263K brains showed a positive signal in all independent reactions (FIG. 4A). Non-seeded reactions or reactions seeded with NBH from old animals showed no positive signals in PMCAb (FIG. 4B). These results are consistent with the previous studies where brain material diluted 10¹²-fold detected PrP^{Sc} and showed stochastic behavior consistent with a limiting dilution of the signal (Id.; Gregori et al. Lancet 364: 529-531 (2004)). In the current experiments, PMCAb achieved the same level of sensitivity as PMCA in 1/7^{th} of the time and with no evidence of spontaneous conversion from NHB substrate.

### Beads Improve the Amplification Rate of a Synthetic Prion Strain

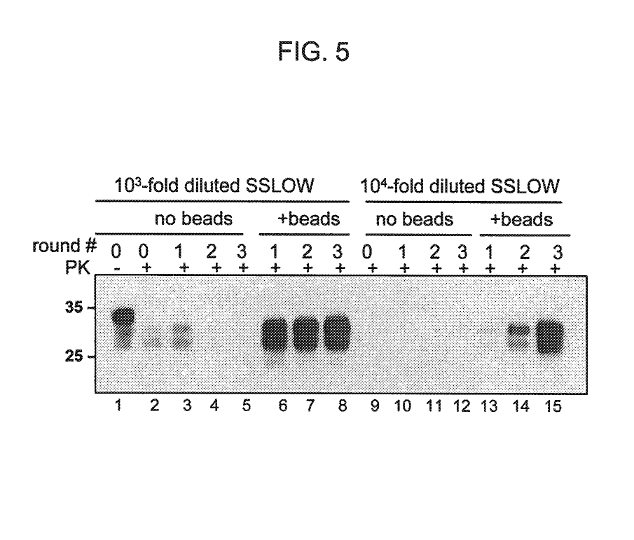

To test whether the positive effect of beads on prion amplification was limited to 263K, we used a synthetic prion strain, SSLOW, which was previously found to have a very peculiar amplification behavior in PMCA (Makarava et al. Acta Neuropathol 119: 177-187 (2010). Previously we found that amplification efficiency of SSLOW varied significantly from preparation to preparation of NBH and that it had a much more unstable amplification behavior than 263K. For instance, SSLOW failed to amplify even in those preparations of NBHs, where 263K showed high amplification rates. In such preparations of NBHs, the amplification fold for SSLOW was found to be lower than the 10-fold dilution factor used for serial PMCA. Therefore, in the absence of beads, SSLOW PrP^{Sc} was no longer detectable by Western blotting after the first round of PMCA (FIG. 5, lanes 3-5). In the presence of beads, however, the amount of SSLOW PrP^{Sc} remained stable during serial PMCAb if the reactions were seeded with 10³-fold diluted SSLOW brain homogenates (FIG. 5, lanes 6-8), or increased if 10⁴-fold dilutions were used for seeding (FIG. 5, lanes 13-15). These results illustrate that the positive effect of beads is not limited to 263K and that beads improved the robustness of PMCA for a strain with poor amplification behavior.

### Beads Counteract the Negative Effect of Recombinant PrP.

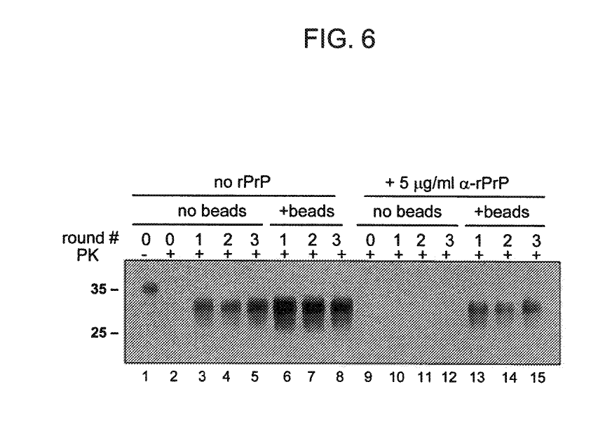

In previous studies, recombinant PrP (rPrP) was found to inhibit amplification of PrP^{Sc} in PMCA (Nishina et al. Biochemistry 45: 14129-14139 (2006)). To test whether the inhibitory effect can be rescued by addition of beads, serial PMCA was performed in the absence or presence of 5 μg/ml Syrian hamster full-length rPrP folded into a α-helical conformation. In the absence of beads, rPrP was found to suppress the amplification of 263K (FIG. 6, lanes 10-12). The addition of beads, however, restored the amplification rate of 263K to the level observed in the absence of rPrP and beads (FIG. 6, compare lanes 13-15 to 3-5). However, this amplification level was lower than those observed in the presence of beads without rPrP (FIG. 6, lanes 6-8).

### Species-Specificity is Preserved in PMCAb.

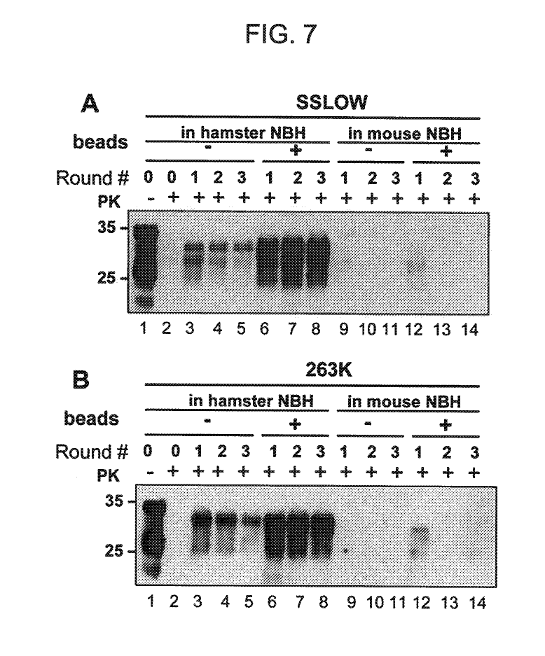

Prion amplification in PMCA was previously shown to exhibit species specificity that faithfully reflects the transmission barrier observed in animals (Castilla et al. Cell 134: 757-768 (2008); Green et al. PLOS Pathog 4: e1000139 (2008)). Considering that beads were found to improve significantly the amplification efficiency, we were interested in testing whether the species specificity was preserved in PMCAb. To address this question, two hamster strains, 263K and SSLOW were used to seed PMCA reactions in mouse NBHs. Consistent with the previous results, beads improved the conversion yield for both strains when they were amplified in Syrian hamster NBH (FIGS. 7A,B). However, when 263K or SSLOW were diluted with mouse NBH no detectible amplification was observed for at least three serial PMCA rounds in the presence or absence of beads (FIGS. 7A,B). A control experiment revealed that mouse RML strain could be amplified in mouse NBH (data not shown, and FIG. 8B). Therefore, the lack of detectible amplification of hamster strains in serial PMCA in mouse NBH confirmed that the presence of beads does not eliminate the species barrier. Taken together, these results illustrate that significant improvements in amplification efficiency do not come at the expense of amplification specificity.

### Effect of Bead Material on Efficiency of Amplification.

To test whether efficiency of amplification depends on the bead material, beads made from eight different materials including Teflon beads purchased from two companies were used for amplification of 10⁵-fold diluted 263K or 10⁴-fold diluted RML (FIGS. 8A,B). Beads made from Teflon and acetal showed the best amplification efficiency for both strains. Nylon and EPDM beads showed very good performance in amplifying RML, but were less efficient for 263K. Notably, the ranking orders in amplification efficiency for different materials appeared to be strain- or species-dependent.

Sonication with Beads Fragments Amyloid Fibrils of rPrP into Small Pieces.

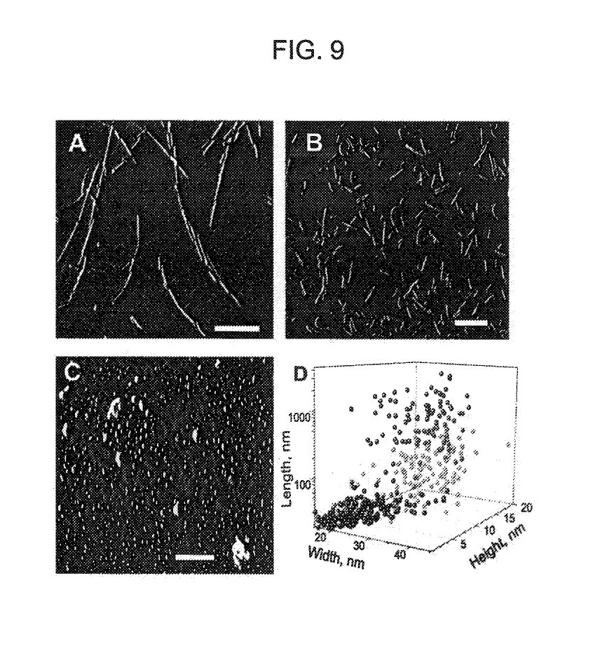

To gain insight into the effect of beads on prion amplification, we tested whether beads affect the fragmentation efficiency of PrP aggregates during sonication. Amyloid fibrils produced from rPrP were sonicated in the presence or absence of beads, and the size of fibrillar fragments was analyzed using atomic force microscopy (AFM) imaging. Consistent with our previous studies (Sun et al. J Mol Biol 376: 11554167 (2008)), sonication was found to break fibrils into smaller fragments (FIGS. 9A,B). Sonication in the presence of beads, however, reduced the size of fibrillar fragments even more producing smaller particles (FIGS. 9C,D). In fact, AFM imaging revealed that after sonication with beads, the fibrillar fragments appeared as small oligomers.

The current studies demonstrated that the yield and the rate of prion conversion in PMCA can be substantially improved by including beads. Remarkably, substantial improvements in the amplification efficiency and robustness did not come at the cost of prion replication specificity. While beads were found to increase the amplification rate of two hamster strains in hamster NBHs, no detectible amplification of these strains were observed in mouse NBHs within three rounds. This shows that the species specificity was preserved (FIG. 7). Furthermore, beads were found to help counteract the negative effect of rPrP on amplification. It is tempting to speculate that the PMCAb format will help to improve the sensitivity of prion detection in body fluids such as blood or urine that might contain inhibitory compounds. Considering substantial enhancement in amplification yield, efficiency and robustness, PMCAb is a promising new platform for developing sensitive and rapid tests for prions, and producing PrP^{Sc} in vitro for structural studies.

The effect of beads on prion amplification can be explained by several mechanisms. Using amyloid fibrils produced from rPrP, we showed that sonication in the presence of beads effectively fragmented rPrP fibrils into pieces that were substantially smaller than those observed in the absence of beads (FIG. 9). This result suggests that beads might improve the efficiency of PrP^{Sc} fragmentation. Consistent with this mechanism, beads were found to enhance significantly the amplification efficiency of SSLOW PrP^{Sc}, a strain which is deposited in the form of large plaques (Makarava et al. Acta Neuropathol 119: 177-187 (2010)). Sonication may not only fragment PrP^{Sc} particles but could also irreversibly damage or denature PrP^{Sc} and/or PrP^{C}. We observed that during sonication, the beads rose from the bottom of the tubes and vibrated in the reaction mixtures. Perhaps, the presence of beads helps to redistribute the cavitation energy of bubbles into the much “softer” energy of mechanical vibration, making the conditions for breaking PrP^{Sc} particles more optimal.

In addition to more efficient fragmentation of PrP^{Sc} particles, the effect of beads could be attributed to a breakage of cellular debris and an increase in the accessibility of PrP^{C} and/or cellular cofactors essential for conversion. Considering that different strains or strains from different species might utilize a variety of cellular cofactors of different chemical natures (Deleault et al. Biochemistry 49: 3928-3934 (2010)), optimizing PMCA amplification might require a different bead material for some strains. Nevertheless, it is currently not known whether any of the proposed mechanisms provides an actual physical explanation for the effect of beads, which at this time should be considered empirical. While the mechanism of bead-induced effect remains to be elucidated in future studies, the PMCAb format offers immediate practical benefits.

In previous studies, only a small subtraction of PrP^{C} could be converted into PrP^{Sc} in PMCA, which raised concerns that only a fraction of PrP^{C} is susceptible to conversion. In an attempt to improve the conversion yield, an increase in the number of PMCA cycles (Saa et al. J Biol Chem 281: 35245-35252 (2006)) or the application of NBH from transgenic mice with high expression of endogenous PrP^{C} was employed (Mays et al. Biochem Biophys Res Commun 388: 306-310 (2009); Kurt et al. J Virol 81: 9605-9608 (2007)). However, it is unclear whether these approaches can favorably change the balance between productive conversion and the competing reactions, which might include spontaneous oxidative modification of PrP^{C} (Breydo et al. Biochemistry 44: 15534-15543 (2005)), the self-cleavage of PrP^{C} (Ostapchenko et al. J Mol Biol 383: 1210-1224 (2008)) and unproductive misfolding. Increasing the time of a PMCA round or the concentration of a substrate is likely to impact both productive and unproductive pathways. The current work shows that an alternative approach that relies on a simple technical modification in the reaction format could be much more rewarding than biochemical approaches. An increase in the conversion yield suggested that beads selectively accelerate the rate of productive conversion of PrP^{C} into PrP^{Sc} without affecting competing reactions. Remarkably, a substantial fraction if not 100% of PrP^{C} could be converted into PrP^{Sc} in PMCAb (FIG. 1). This result argues that the amplification yield is not limited to a small subfraction of PrP^{C} susceptible to conversion or by cellular cofactors involved in the conversion reactions.

The most beneficial effect of beads on amplification was observed at high seed dilutions, i.e. at high PrP^{C}/PrP^{Sc} ratios when the supply of PrP^{C} was unlimited (FIG. 3 and FIG. 13). In this case, beads improved the sensitivity of detection by at least 2 or 3 orders of magnitude. When seeded with high concentrations (10³ or 10⁴-dilution of scrapie brain material), the differences in amplification yield between PMCA and PMCAb was approximately 10-fold (FIG. 1). A 10-fold difference was also consistent with the difference in mean incubation time observed between the two groups after inoculation (FIG. 2). However, this difference must be considered tentative due to the low statistical significance of this measurement. Regardless, the bioassay confirmed that PMCAb amplifies prion infectivity at least equivalently to PMCA.

In our experience, prion amplification in PMCA is very sensitive to technical settings such as the precise position of a tube within the microplate horn, i.e. the distance of a tube from horn's surface and its center; the age of the sonicator's horn; the tube's shape. Furthermore, aging of sonicatior's horn and individual patterns of horn erosion with age cause time- and position-dependent variations in sonication power. As a result, it is difficult to obtain consistent amplification of PrP^{Sc} in experiments performed in different sonicators or even using the same sonicator as it ages. For instance, the differences in the yield of PrP^{Sc} amplification seen in lanes B6 and A1 in FIG. 1 were attributed to the aging of the sonicator's horn, as both these experiments were performed using the same sonicator but at a slightly different age. In our experience, Teflon beads significantly improve the robustness of PMCA making prion amplification less sensitive to technical variations, which are difficult to control. The new format should help to establish a PMCA-based approach for assays of prion infectivity.

### Example 2

### Fast and Ultrasensitive Method for Quantitating Prion Infectivity Titer.

Bioassay by end-point dilution titration has been employed for decades for routine determinations of relative concentrations of priori infectivity. Here we show that the new Protein Misfolding Cyclic Amplification format with beads (PMCAb) can used to obtain a titer with a higher level of precision, with 10²-10³ fold greater sensitivity, and in 3 to 6 days as opposed two years, compared with bioassay. Differences in sensitivity between bioassay- and PMCAb-based titers were found to be strain-specific and presumably reflect the strain-specific differences in the number of PMCAb active particles required for infecting an animal.

The traditional method for obtaining a quantitative estimate of prion infectivity is end-point dilution titration in animals. A suspension of the tissue or fluid of interest is diluted in ten-fold serial steps and then each dilution is inoculated into a group of animals. A dilution at which only a fraction of the inoculated animals develop clinical signs of disease or show positive evidence of PrP^{Sc} on immunoassay is called a “limiting dilution.” At limiting dilution there are only one or a few infectious doses per inoculation volume. End point dilution titers are typically expressed as infectious dose 50 (ID₅₀): the reciprocal of the dilution required to infect only 50% of the animals inoculated as determined by interpolation or other statistical methods. While the end-point bioassay has been the principal method for determining prion infectivity, the assay is extremely long, expensive and laborious. Moreover, the bioassay works optimally only for prion strains with incubation times well within the life span of the host.

A pathognomonic hallmark of the prion diseases is the accumulation of misfolded isoform of the prion protein, PrP^{Sc}. Alternatives to end-point titration are biochemical, immunochemical or cell culture assays that assess either the presence, mass or concentration of PrP^{Sc} (Safar et al. Nat. Med. 4, 1157-1165 (1998); Safar et al. Nat. Biotechnol. 20, 1147-1150 (2002); Wadsworth et al. Lancet 358, 171-180 (2001); Klöhn et al. Proc. Acad. Natl. Sci. U.S.A. 100, 11666-11671 (2003)). However, establishing accurate quantitative relationships between PrP^{Sc} concentration and prion titer has proven to be difficult because of the size heterogeneity of prion particles and uncertainty over whether all prion particles is equally infectious (Silveira et al. Nature 437, 257-261 (2005)), Moreover, the size distribution and physical properties of prion particles appear to vary with agent strain and host species.

Protein Misfolding Cyclic Amplification (PMCA) propagates prion infectivity in vitro (Saborio et al. Nature 411, 810-813 (2001)). The sensitivity of the PMCA reaction to detect prion particles exceeds that of the bioassay to detect a single unit of infectivity (Saa et al. J. Biol. Chem. 281, 35245-35252 (2006); Chen et al. Nat Methods 7, 519-520 (2010)). The improvements in the PMCA assay found in PMCAb have resulted in a much faster, more robust, sensitive and cost-efficient way of measuring PrP^{Sc} compared with either PMCA or bioassay (Gonzalez-Montalban et al. PLoS Pathogen 7, e1001277 (2011)). To illustrate the advantages of PMCAb based end point titration, we assessed the relative concentrations of PrP amyloid in brain material of two rodent strains, 263K and SSLOW, which display very short or very long incubation time to symptomatic disease, respectively (Makarava et al. Acta Neuropathol. 119, 177-187 (2010)).

For establishing a prion amyloid titer, 10% brain homogenates prepared from 263K or SSLOW-infected animals were diluted in ten-fold serial steps, then aliquots from each dilution were used to seed serial PMCAb reactions. Up to ten independent serial PMCAb reactions were conducted for each dilution for each strain. Each PMCAb round consisted of 48 cycles, 30 min each. Three or six serial PMCAb rounds were sufficient for amplification of even the highest dilutions of 263K or SSLOW, respectively, to the level detectible by Western blot. An increase in number of PMCAb rounds did not increase the percentage of positive reactions for the most highly diluted samples illustrating that limiting dilution was reached.

In parallel to PMCAb titration, the infectivity titers of brains from 263K and SSLOW-infected animals were measured using end-point dilution bioassay. Animals were considered infected if they develop symptomatic disease or if their brains contained PrP^{Sc} as judged by Western blot even without symptomatic disease. The fractions of animals infected at each dilution are presented in Table 1.

TABLE 1
Summary of end-point titration of SSLOW

| Dilution of | Clinical TSE/total | Positive by Western |
|---|---|---|
| brain material | inoculated | blot/total inoculated |
| 10¹ | 8/8 | — |
| 10² | 8/8 | — |
| 10³ | 8/8 | — |
| 10⁴ | 8/8 | — |
| 10⁵ | 5/5 | 5/5 |
| 10⁶ | 4/8 | 8/8 |
| 10⁷ | 0/8 | 5/8 |
| 10⁸ | 0/8 | 2/8 |
| 10⁹ | 0/8 | 0/8 |
| 10¹⁰ | 0/6 | 0/6 |
| 10¹¹ | 0/8 | 0/8 |

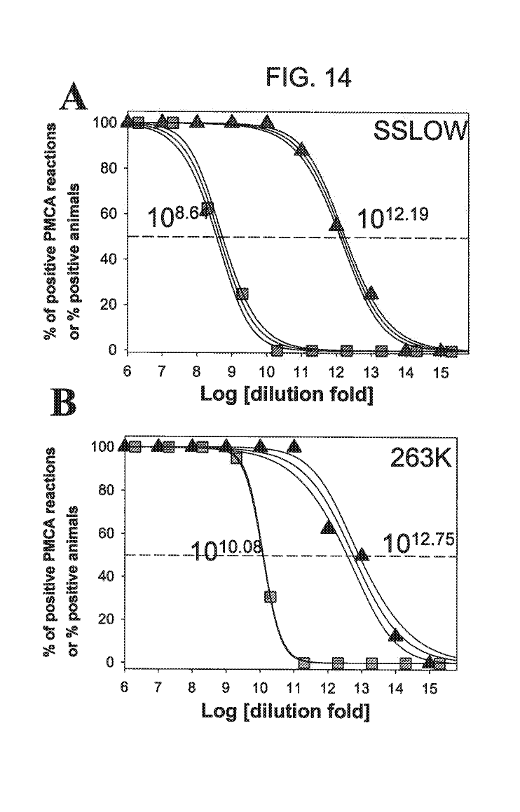

For each dilution, the fractions of animals infected, or PMCAb reactions with a positive signal on Western blot, were plotted against the logarithm of dilution for both 263K and SSLOW (FIGS. 14A and B). Non-linear regression of the data to a sigmoidal function was used to calculate ID₅₀ or PMCAb₅₀ values from bioassay- or PMCAb-based end point curves, respectively (Table 2). The infectivity titers determined in this way were nearly identical to those determined by the Reed and Meunch, and Spearman and Karber methods. Analogous to the bioassay, a PMCAb₅₀ is the reciprocal of the concentration at which only 50% of the PMCAb reactions amplify PrP^{Sc}. As judged from ID₅₀ and PMCAb₅₀ values, PMCAb was more sensitive than bioassay by ˜3,500 fold for SSLOW and nearly 500 fold in case of 263K. Importantly, PMCAb titration was completed in few days, whereas bioassay of SSLOW required nearly two years.

TABLE 2
ID₅₀ values measured by end-point bioassay or PMCAb

| | Assay₅₀ units/g of tissue | | |
|---|---|---|---|
| | Bioassay | PMCAb | |
| | ID₅₀ | PMCAb₅₀ | PMCAb₅₀/ID₅₀ |
| 263K | 10^{10.08} | 10^{12.75} | 470 |
| SSLOW | 10^{8.64} | 10^{12.19} | 3550 |

A PMCAb₅₀ titer represents the number of PrP^{Sc} particles capable of initiating a PMCAb amplification per gram of material. As judged from PMCAb titration, the concentration of PrP^{Sc} particles was very similar in brains of animals infected with 263K or SSLOW despite that 263K (short incubation, short clinical duration) and SSLOW (very long incubation and clinical duration) represent two extremes of prion hamster disease (Makarava et al. Acta Neuropathol. 119, 177-187 (2010)). These results suggest either that PrP^{Sc} accumulates more slowly in SSLOW-infected animals and/or that SSLOW PrP^{Sc} particles are substantially less toxic than that of 263K.

The ratio between the two endpoints, one measured by bioassay and the other from PMCAb, was different for two strains (Table 2). One can interpret the PMCAb₅₀/ID₅₀ ratio as the minimum number of PMCAb active particles required for infecting an animal via particular route, i.e. the specific infectivity relative to PMCAb reactive material. Therefore, the difference in the PMCAb₅₀/ID₅₀ ratio presumably reflects the strain-specific differences in the amount of PMCAb active particles required for infecting an animal, i.e. the efficiency of infection for any given particle is ˜10 fold greater for 263K than for SSLOW. However, we do not know whether we would have eventually seen ID₅₀ for SSLOW equivalent to that of 263K, if hamsters have had longer life span.

Soto and coworkers, estimated the amount of PrP^{Sc} based on the number of PMCA rounds necessary to amplify prions to a detectible level (Chen et al. Nat Methods 7, 519-520 (2010)). While PMCAb and PMCA methods have similar sensitivity, for 263K PMCA requires 18 days to reach sensitivity comparable to that achieved by PMCAb in 3 days. PMCAb is also far less sensitive to inhibitors and small variations in sonication conditions that have plagued conventional PMCA.

We show here that PMCAb can be used to obtain an estimate of prion infectivity titer in only 3 to 6 days, approximately 100 times faster than the bioassay. At the same time, PMCAb is two to three orders of magnitude more sensitive than bioassay. The precision of the measurements in PMCAb is limited only by the number of replicates performed. The new PMCAb platform can be used as a fast, efficient and ultrasensitive method for determining prion titer, and is uniquely beneficial for samples that have extremely low levels of infectivity and for determining infectivity concentrations for prion strains with long incubation times.

### Methods

### Endpoint Titration Bioassay

10% scrapie BH was prepared in PBS, pH 7.4 by sonication and serially diluted to up to 10¹¹ fold in PBS as previously described (Gregori et al. Transfusion 46, 1152-1161 (2006)). SSLOW-inoculated animals from the second passage of SSLOW (Makarava et al. Acta Neuropathol. 119, 177-187 (2010)) were used for both bioassay and PMCAb. Before inoculation, samples were dispersed by 90 seconds of maximum power ultrasonication in PBS. Each hamster received 50 μl inoculum intracerebrally under general anesthesia (2% O2/4 MAC isoflurane). The inoculated animals were observed closely up to 660 days post inoculation or until they developed clinical signs of prion disease. For SSLOW-inoculated animals, the clinical signs were observed as early as 315±8 days post inoculation for the 10 fold diluted or as late as 560±8 days for the 10⁶ fold diluted brain material. No clinical sings were observed for the dilutions 10⁷ fold and above. Affected animals were euthanized and their disease status was confirmed by Western blot analysis of their brains. At the end of the incubation (660 days post inoculation) all remaining animals were euthanized and all brains were assessed for the presence PrP^{Sc} by Western blot. Animals, whose brains contained PrP^{Sc} but had not yet developed symptomatic disease were considered infected.

### End-Point Titration Using PMCAb

Healthy hamsters were euthanized and immediately perfused with PBS, pH 7.4, supplemented with 5 mM EDTA. Brains were dissected, and 10% brain homogenate (w/v) was prepared using ice-cold conversion buffer and glass/Teflon tissue grinders cooled on ice and attached to a constant torque homogenizer (Heidolph RZR2020). The brains were ground at low speed until homogeneous, then 5 additional strokes completed the homogenization. The composition of conversion buffer was as previously described (Castilla et al. Cell 121, 195-206 (2005)); Ca²⁺-free and Mg²⁺-free PBS, pH 7.4 supplemented with 0.15 M NaCl, 1.0% Triton and 1 tablet of Complete protease inhibitors cocktail (Roche, Cat. #1836145) per 50 ml of conversion buffer. The resulting 10% normal brain homogenate (NBH) in conversion buffer was used as the substrate in PMCA reactions. To prepare seeds, scrapie infected brains were homogenized as for inoculation (above) and 100 ml aliquots were sonicated in MISONIX S-400 microplate horn for 30 sec at 50% power before serial dilution from 10- to 10¹⁴-fold in conversion buffer. Ten, 10 μl of each dilution was used to seed 90 μl of NBH in PMCAb. Teflon beads (2.38 mm diameter, McMaster-Carr, Los Angeles, Calif.) were placed into the 0.2 ml tubes first, then NBH and seeds were added. Samples in 0.2 ml PCR tubes (Fisher, Cat. #14230205) were placed in a floating rack inside a Misonix S-4000 microplate cup horn filled with 350 ml water. Two coils of rubber tubing attached to a circulating water bath were installed for maintaining 37° C. inside the sonicator chamber. The standard sonication program consisted of 30 sec sonication pulses delivered at 50% efficiency applied every 30 min during a 24 hour period. For each subsequent round of serial PMCAb, 10 μl aliquotes from a previous round were used to seed the reactions in the next round. At limiting dilutions, up to ten independent serial PMCAb reactions were used for each dilution to accumulate statistics.

### Data Analysis

For titrating prion infectivity, 100 μl reaction volume was used for PMCAb and 50 μl inoculum volume was used for bioassay. Therefore, the fractions of positive PMCAb reactions or positive animals presented in FIG. 1 were normalized per gram of brain tissue. To determine ID₅₀ and PMCAb₅₀ values, regression analysis in Sigma Plot was used for both sets of data, obtained from PMCAb or bioassay end-point titrations. Nonlinear least-square fitting of the data to the following equation was performed:

F=(100*exp(−(A+B*x)))/(1+exp(−(A+B*x)))

where F is percent of positive PMCAb reactions or infected animals, x is logarithm of the dilution fold, A and B are two fitting parameters that define the position of a limiting dilution transition on the x axis and the slope of the transition, respectively. ID₅₀ and PMCAb₅₀ were calculated according to the equation: PMCAb₅₀ or ID₅₀=A/B

### Proteinase K Assay

To analyze PMCAb end point titration reactions, 10 μl of each sample was supplemented with 5 μl SDS and 5 μl PK, to a final concentration of SDS and PK of 0.25% and 50 μg/ml respectively, followed by incubation at 37° C. for 1 hour. The digestion was terminated by addition of SDS-sample buffer and boiling thr 10 min. Samples were loaded onto NuPAGE 12% BisTris gels, transferred to PVDF membrane, and stained with 3F4 antibody.

To analyze scrapie brain homogenates, an aliquot of 10% brain homogenate was mixed with an equal volume of 4% sarcosyl in PBS, supplemented with 50 mM Tris, pH 7.5, and digested with 20 μg/ml PK for 30 min at 37° C. with 1000 rpm shaking (Eppendorf thermomixer). The reaction was stopped by SDS sample buffer. Samples were boiled for 10 min and loaded onto NuPAGE 12% BisTris gels. After transfer to PVDF membrane, PrP was detected with 3F4 antibody.

All patents and publications mentioned and/or cited herein are incorporated by reference to the same extent as if each individual publication was specifically and individually indicated as having been incorporated by reference in its entirety.

1. A method for amplifying and detecting PrP^{Sc} in a sample, comprising:
i) contacting the sample with a source of PrP^{C} to make a reaction mixture;
ii) incubating the reaction mixture;
iii) agitating the reaction mixture of (ii) in the presence of one or more beads; and
iv) detecting the amplified PrP^{Sc}.

2. The method of claim 1, wherein steps (ii) and (iii) are repeated between 1 and 500 times before step iv) is conducted.

3. The method of claim 1, wherein a portion of the reaction mixture is diluted and added to additional PrP^{C} after a number of steps of ii) and iii) have been performed, and further repeating steps ii) and iii) one or more times with the diluted sample.

4. The method of claim 1, wherein the reaction mixture is incubated for a period of time selected from the group consisting of: approximately 5 minutes, approximately 10 minutes, approximately 15 minutes, approximately 20 minutes, approximately 25 minutes, approximately 30 minutes, approximately 35 minutes, approximately 40 minutes, approximately 45 minutes, approximately 50 minutes, approximately 55 minutes, approximately 60 minutes, approximately 75 minutes, approximately 90 minutes and approximately 120 minutes.

5. The method of claim 1, wherein the reaction mixture is agitated by sonication.

6. The method of claim 1, wherein the one or more beads are made from a substance selected from the group consisting of: one or more polymeric substances, polytetrafluoroethylene (PTFE; TEFLON), stainless steel, neoprene, nylon, ethylene propylene diene monomer (EPDM), nitrile rubber, zytel nylon, acetal, glass, ceramic, polypropylene and polystyrene.

7. The method of claim 1, wherein the beads are at least a size selected from the group consisting of: about 0.1 mm, about 0.2 mm, about 0.3 mm, about 0.4 mm, about 0.5 mm, about 0.6 mm, about 0.7 mm, about 0.8 mm, about 0.9 mm, about 1 mm, about 1.1 mm, about 1.2 mm, about 1.3 mm, about 1.4 mm, about 1.5 mm, about 1.59 mm, about 1.6 mm, about 1.7 mm, about 1.8 mm, about 1.9 mm, about 2.0 mm, about 2.1 mm, about 2.2 mm, about 2.3 mm, about 2.38 mm, about 2.4 mm, about 2.5 mm, about 2.6 mm, about 2.7 mm, about 2.8 mm, about 2.9 mm, about 3.0 mm, about 3.5 mm, about 4.0 mm, about 4.5 mm and about 5.0 mm.

8. The method of claim 1, wherein the number of beads is selected from the group consisting of: one bead, 2 beads, 3 beads, 4 beads, 5 beads, 6 beads, 7 beads, 8 beads, 9 beads, 10 beads, 11 beads, 12 beads, 13 beads, 14 beads, 15 beads, 16 beads, 17 beads, 18 beads, 19 beads, 20 beads, 21 beads, 22 beads, 23 beads, 24 beads and 25 beads or wherein the volume occupied of the total reaction mixture by the one or more beads is selected from the group consisting of: 10%, 20%, 30%, 40% and 50%.

9. The method of claim 1, wherein the fold amplification of PrP^{Sc} achieved in the presence of the one or more beads is about 10-fold to about 1000-fold greater than amplification without the bead(s) present.

10. The method of claim 1, wherein the source of PrP^{C} is from an origin selected from the group consisting of: human, bovine, ovine, hamster, rat, mouse, canine, feline, goat, cervid, and non-human primate.

11. The method of claim 1, wherein the PrP^{C} is from a source selected from the group consisting of a tissue sample (whole tissue, homogenate or fraction thereof), a bodily fluid sample, cell lysate from cultured cells, a recombinant source and a transgenic animal.

12. The method of claim 1, wherein the source of PrP^{C} is normal brain homogenate.

13. The method of claim 1, wherein the sample is from an organism suspected of, at risk of, or known to have a prion disease, wherein the prion disease is selected from the group consisting of: scrapie (typical and atypical forms) in sheep, bovine spongiform encephalopathy (BSE; also known as mad cow disease, including classical and the atypical forms BSE-H and BSE-L) in cows, bovine amyloidotic spongiform encephalopathy (BASE) in cows, transmissible mink encephalopathy (TME) in mink, chronic wasting disease (CWD) in elk, moose, or deer, feline spongiform encephalopathy, ungulate encephalopathy in nyala, oryx or greater kudu, and Creutzfeldt-Jakob disease (CJD) and its varieties (including but not limited to iatrogenic Creutzfeldt-Jakob disease (iCJD), variant Creutzfeldt-Jakob disease (vCJD), genetic Creutzfeldt-Jakob disease (fCJD), and sporadic Creutzfeldt-Jakob disease (sCJD)), Gerstmann-Straussler-Scheinker syndrome (GSS), fatal familial insomnia (fFI), sporadic fatal insomnia (sFI), kuru, and Alpers syndrome in humans.

14. The method of claim 1, wherein the detection of PrP^{Sc} indicates the presence of a prion disease.

15. The method of claim 1, wherein the sample is from an organism selected from the group consisting of human, bovine, cervids, sheep, primate and rodent.

16. The method of claim 1, wherein the sample is a tissue sample (whole tissue, homogenate or fraction thereof) or other sample of bodily origin including, but not limited to, blood, lymph nodes, brain tissue (includes whole brain, anatomical parts, or fractions and homogenates thereof), spinal cord, tonsils, internal organs (such as spleen, stomach, pancreas, liver, intestine (large or small), lungs, heart, thymus, bladder or kidney), skin, muscle, appendix, olfactory epithelium, nasal tissue, cerebral spinal fluid, urine, feces, milk, mucosal secretions, tears and/or saliva.

17. A method of screening to identify an agent that modulates PrP^{Sc} formation, comprising
i) contacting a sample having PrP^{Sc} with a source of PrP^{C} to make a reaction mixture;
ii) incubating the reaction mixture in the presence and absence of the agent;
iii) agitating the reaction mixture of (ii) in the presence of one or more beads; and
iv) detecting and comparing the level of the amplified PrP^{Sc} generated in the presence and absence of the agent.

18. The method of claim 17, wherein steps (ii) and (iii) are repeated between 1 and 500 times before step iv) is conducted.

19. A kit for the amplification and detection of PrP^{Sc}, wherein the kit comprises in a suitable container, one or more beads, a source of PrP^{C}, and optionally further comprises one or more of the following components: 1) a reaction mixture buffer; 2) decontamination solution; 3) a positive control sample containing PrP^{s}% 4) a negative control sample that does not contain PrP^{Sc}; 5) one or more proteases; and 6) one or more reagents for the detection of PrP^{Sc}.
